(19) United States
(12) Patent Application Publication
(10) Pub. No.: US 2026/0314952 A1
(43) Pub. Date: Oct. 15, 2026

(54) SYSTEMS AND METHODS FOR ELECTROCHEMICAL DETECTION OF DRUGS IN GASEOUS SAMPLES

(71) Applicant: Ramot at Tel-Aviv University Ltd., Tel-Aviv (IL)

(72) Inventors: Fernando PATOLSKY, Tel-Aviv (IL); Boris FILANOVSKY, Tel-Aviv (IL); Vadim KRIVITSKY, Tel-Aviv (IL); Guy DAVIDI, Tel-Aviv (IL)

(73) Assignee: Ramot at Tel-Aviv University Ltd., Tel-Aviv (IL)

(21) Appl. No.: 19/466,431

(22) Filed: Feb. 1, 2026

Related U.S. Application Data
(63) Continuation of application No. PCT/IL2024/050768, filed on Aug. 1, 2024.
(60) Provisional application No. 63/530,082, filed on Aug. 1, 2023.

(51) Int. Cl. A61B 5/08; A61B 5/00; A61B 5/097; G01N 27/30; G01N 27/413

(52) U.S. Cl. CPC A61B 5/082; A61B 5/097; A61B 5/4845; G01N 27/308; G01N 27/413

(57) ABSTRACT

A method of detecting a presence, amount and/or type of an electroactive (electrochemically detectable) drug such as cannabinoids and amphetamines in a gaseous sample is provided. The method is effected by contacting the sample with a carbon electrode; applying potential to the electrode; and measuring an electrochemical parameter of the carbon electrode, wherein the electrochemical parameter is indicative of a presence and/or amount and/or type of the electroactive drug in the sample. Carbon electrodes, electrochemical cells comprising same and devices or systems comprising the electrochemical cells, which are configured for detecting electroactive drugs in a gaseous sample, are also provided.

### RELATED APPLICATIONS

This application is a Continuation of PCT Patent Application No. PCT/IL2024/050768 filed on Aug. 1, 2024 which claims the benefit of priority under 35 USC § 119(e) of U.S.

Provisional Patent Application No. 63/530,082, filed on Aug. 1, 2023. The contents of the above applications are all incorporated by reference as if fully set forth herein in their entirety.

### FIELD AND BACKGROUND OF THE INVENTION

The present invention, in some embodiments thereof, relates to electrochemical sensing and, more particularly, but not exclusively, to a system and method for electrochemically detecting a presence, amount and/or type of a drug in a gaseous sample.

The use of illegal and/or recreational drugs is a worldwide phenomenon that affects hundreds millions of people. Amongst other risks associated with the use of such drugs is the altered state of consciousness, which may put the user and the society in danger under certain circumstances, such as, for example, when driving.

Recreational drugs include, inter alia, alcohol, cannabis and hashish, nicotine, caffeine, amphetamine, heroin, cocaine, LSD, psilocybin mushrooms, MDMA, club drugs and some prescription drugs. Most of these drugs are generally illegal while some are illegal only when used under certain circumstances such as driving.

In the field of forensic chemistry, increasing roadside/on-site drug screening availability is critical for the fight against trade and use of illegal drugs. Roadside testing for drugs of abuse has a number of requirements: it needs to be fast, ideally 15-30 seconds (e.g., as in a breath alcohol test), very sensitive, ideally capable of detecting less than 10 ng per sample, it should preferably be non-invasive, with built-in controls, difficult to tamper with and be portable. A further important criterion is that the test must be easy to perform by non-laboratory personnel.

Currently available drug screening products require a minimum of 5-9 minutes for a test [see, for example, U.S. Patent Application Publication No. 2011/0151570]. Test time and cost are currently restricting the roadside drug screening market to less than 10% the volume of the alcohol screening market [Wanklyn et al., Chem. Cent. J. 2016, 10, 1].

Several methodologies have been reported for detecting recreational drugs. These are based on, for example, electrochemistry, mass spectrometry, infra-red spectrometry, Raman spectrometry, X-ray diffractometry, ion mobility spectrometry, gas/liquid chromatography, UV-vis spectrometry, colorimetric test, immunoassay, urine dipstick test, and other methods [Harper et al., Harm. Reduct. J. 2017, 14, 52; Beck et al., J. Anal. Toxicol. 2012, 36, 638-646; and Trefz et al., J. Breath Res. 2017, 11, 024001]. These methods typically involve time-consuming procedures, high costs and/or operation by well qualified staff, which limits their application in field conditions.

Additional methods rely on trained animals and utilize their highly developed sense of smell to detect drugs. These methods, however, require intense and expensive training of the animals, and handling by an expert.

Electrochemical detection is a powerful analytical method that can detect electric currents generated from oxidative or reductive reactions in test compounds. The method utilizes electrodes, immersed in an electrolyte medium, and connected to a potentiostat, which controls the voltage difference between the electrodes. Normally, during an electrochemical reaction the electrode potential is varied and the generated electric current between the electrodes is characteristic of the presence of an electrochemically reactive compound in the electrolyte. Electrochemical detection methods and devices are typically highly sensitive, relatively simple, cost-effective and reliable.

Recreational drugs such as cannabinoids and amphetamines are electrochemically active compounds.

Over 100 chemical compounds are found in cannabis or marijuana plant, and are known as cannabinoids. The most popular and important cannabinoids include Tetrahydrocannabinol (THC), tetrahydrocannabinolic acid (THCA), cannabidiol (CBD) and cannabidiolic acid (CBDA).

The most popular and important amphetamines include methamphetamine, ephedrine, cathinone, phentermine, mephentermine, bupropion, methoxyphenamine, selegiline, amfepramone, pyrovalerone, and MDMA.

Electrochemical methods for detecting these drugs, which typically involve oxidation of the target analyte, are described, for example, in a review by Zanfrognini et al., J. Solid State Electrochem., 2020.

These electrochemical tests may run through cyclic voltammetry (CV), differential pulse voltammetry (DPV), or square-wave voltammetry (SWV) modes. Different kinds of working electrodes can be utilized in those detection methods. In particular, carbon-based sensors use carbon at the working electrode, including screen-printed electrodes (SPE), glassy carbon electrodes (GCE), and carbon paper electrodes [Klimuntowski et al., ACS Sens. 2020, 5, 620-636]. Some of the disclosed working electrodes are modified with small molecules, (bio)macro-molecules, and/or inorganic nano-materials [Naghian et al., New J. Chem. 2020, 44, 9271-9277; Florea et al., Curr. Opin. Electrochem. 2018, 11, 34-40; and Stevenson et al., Sci. Rep. 2019, 9, 12701].

Electrochemical tests for detecting cannabinoids, and THC in particular, using carbon electrodes of different types and chemical nature, have been described, for example, in U.S. Pat. No. 9,011,657; Wanklyn et al., Chem. Cent. J. 2016, 10, 1; Balbino et al., J. Solid State Electrochem. 2016, 20, 2435-2443; and Renaud-Young et al., Electrochimica Acta 2019, 307, 351-359. All of these tests use saliva samples.

Electroanalytical sensing of Rohypnol (flunitrazepam) was reported regarding the use of screen-printed graphite electrodes without the requirement for any additional pre-treatment or modification [Smith et al, Analyst 2013, 138, 6185-6191]. The methodology is shown to be useful for quantifying low levels (1 mg/mL) of Rohypnol in two internationally favored drinks: Coca Cola and the alcopop WKD, without any sample pre-treatment. Cyclic voltammetry method was used to detect the drug in beverages upon the addition of a buffering salt.

A bare carbon-base electrode was reported to selectively detect heroin in street samples [Florea et al., Anal. Chem. 2019, 91, 7920-7928]. Heroin, mixing agents (adulterants, cutting agent, and impurities), and their binary mixtures were subjected to square-wave voltammetry measurements at bare graphite electrodes at pH 7.0 and pH 12.0, in order to elucidate a unique electrochemical fingerprint of heroin and mixing agents as well as possible interferences or reciprocal influences.

Facilitation of on-site testing was demonstrated in different cases. For example, a wearable glove-based sensor that can detect fentanyl electrochemically on the fingertips towards decentralized testing for opioids was reported [Barfidokht et al., Sens. Actuators B Chem. 2019, 296, 126422]. The “Lab-on-a-Glove” sensor consisted of flexible screen-printed carbon electrodes modified with a mixture of multi-walled carbon nanotubes and a room temperature ionic liquid, 4-(3-butyl-1-imidazolio)-1 butanesulfonate. The sensor showed direct oxidation of fentanyl in both liquid and powder forms with a detection limit of 10 μM using square-wave voltammetry.

DE 10335236 describes a measuring system for determining the concentration of propofol in the respiratory using diamond-like carbon electrode and a mediator.

While electrochemical methods for detecting cannabinoids and amphetamines have been described in the literature, most of these methods utilize saliva samples. However, the retention of cannabinoids in the saliva is not correlated with its psychoactive window. Thus, while cannabinoids exhibit a psychoactive effect up to about 3 hours after consumption, they can still be detected in the saliva and other bodily fluids after the psychoactive window, rendering the detection method irrelevant when used to assess the cognitive state of the consumer.

Studies have shown that the levels of cannabinoids in-breath can be as low as pg/mL. One of the most commonly used methods for detecting cannabinoids in breath is liquid chromatography-tandem mass spectrometry (LC-MS/MS) using electrospray ionization, which typically requires chemical modification prior to the detection. See, for example, Luo et al. (2019) Journal of Analytical Toxicology, 2019; 1-9.

Additional examples for relevant drug electrochemical detection devices are described in WO 2016/166623; U.S. Patent Application Publication No. 2011/0102564; and U.S. Pat. Nos. 9,011,657 and 5,656,142.

Additional background art includes WO 2012/077110; WO 2018/229780; WO 2018/229781 and WO 2020/129070.

### SUMMARY OF THE INVENTION

According to an aspect of some embodiments of the invention, there is provided a method of detecting a presence, amount and/or type of an electroactive (electrochemically detectable) drug in a gaseous sample, the method comprising: contacting the sample with a carbon electrode; applying potential to the electrode; and measuring an electrochemical parameter of the sensing electrode, wherein the parameter is indicative of a presence and/or amount and/or type of the electroactive drug in the sample.

According to some of any of the embodiments described herein, the carbon electrode is a gas-permeable electrode and the contacting is by contacting at least a portion of the electrode with the gaseous sample.

According to some of any of the embodiments described herein, the electrode is a carbon fiber electrode.

According to some of any of the embodiments described herein, the electrode is a carbon fiber microelectrode.

According to some of any of the embodiments described herein, the electrode is a carbon paper microelectrode.

According to some of any of the embodiments described herein, the electroactive drug is a recreational drug.

According to some of any of the embodiments described herein, the drug is a cannabinoid.
the drug is selected from tetrahydrocannabinol (THC), tetrahydrocannabinolic acid (THCA), cannabidiol (CBD), cannabinol (CBN), cannabigerol (CBG), cannabichromene (CBC), tetrahydrocannabivarin (THCV), cannabidivarin (CBDV) and cannabidiolic acid (CBDA).

According to some of any of the embodiments described herein, the drug is an amphetamine.

According to some of any of the embodiments described herein, the amphetamine is methamphetamine.

According to some of any of the embodiments described herein, the drug features a vapor pressure of no more than 0.1 Pa, or lower than 0.01 Pa, or lower, at room temperature.

According to some of any of the embodiments described herein, the carbon electrode features at least one functional moiety covalently attached thereto, the functional moiety being capable of interacting with the electroactive drug, and of allowing electron transfer through the electrode.

According to some of any of the embodiments described herein, the functional moiety is a lipophilic moiety.

According to some of any of the embodiments described herein, the functional moiety comprises an electron-withdrawing group, preferably a non-hydrophilic electron-withdrawing group.

According to some of any of the embodiments described herein, the functional moiety comprises a polar group, preferably a non-hydrophilic polar group.

According to some of any of the embodiments described herein, the functional moiety comprises a hydrocarbon of at least 4 carbon atoms substituted by the at least one electron-withdrawing group.

According to some of any of the embodiments described herein, the functional moiety comprises a hydrocarbon of at least 4 carbon atoms substituted by the at least one polar group.

According to some of any of the embodiments described herein, the functional moiety is selected from fluoro-substituted phenyl, and a fluoro-substituted alkylene of at least 4 carbon atoms in length.

According to some of any of the embodiments described herein, the functional moiety is attached to the carbon electrode via a siloxane linking moiety, or via one or more —O—Si— bonds.

According to some of any of the embodiments described herein, the gaseous sample is an air sample.

According to some of any of the embodiments described herein, the air sample is of an environment of the drug (e.g., an intact drug or a heated drug or a smoked drug).

According to some of any of the embodiments described herein, contacting the electrode with the gaseous sample is by means of a pump (e.g., an air pump).

According to some of any of the embodiments described herein, the gaseous sample is a breath sample (e.g., of a subject suspected as consuming the drug).

According to some of any of the embodiments described herein, contacting the gaseous sample with the electrode is by having the subject breathing in the vicinity of the electrode.

According to some of any of the embodiments described herein, the gaseous sample comprises no more than 10 ng/liter of the drug (in a gaseous state).

According to some of any of the embodiments described herein, the carbon electrode forms a part of an electrochemical cell and the electrochemical cell is operable by electrically connecting the electrode to a power source.

According to some of any of the embodiments described herein, the electrochemical cell further comprises a reference electrode and optionally an auxiliary electrode.

According to some of any of the embodiments described herein, applying the potential is by a cyclic voltammetry (CV) mode, a differential pulse voltammetry (DPV) mode, or a square-wave voltammetry (SWV) mode, preferably by cyclic voltammetry.

According to some of any of the embodiments described herein, the electrochemical cell further comprises an electrolyte.

According to some of any of the embodiments described herein, the method further comprising, prior to applying the potential, contacting the electrode with the electrolyte.

According to some of any of the embodiments described herein, the method comprises, subsequent to contacting the electrode with the sample, contacting the electrode with an electrolyte.

According to some of any of the embodiments described herein, the electrochemical cell is connectable to a hardware processor configured for receiving the signal generated by the electrode and processing the signal.

According to some of any of the embodiments described herein, the hardware processor is configured for transmitting the processed signal to a server computer at a remote location.

According to some of any of the embodiments described herein, the hardware processor is configured for processing the signal to determine presence, amount and/or type of the drug or portion thereof on the electrode and to transmit information pertaining to the determination to a server computer at a remote location.

According to some of any of the embodiments described herein, the method further comprising an indication unit in the form of a visual or an audio display.

According to some of any of the embodiments described herein, the contacting is for a time period of no more than 1 minute, or no more than 30 seconds, or no more than 10 seconds.

According to some of any of the embodiments described herein, a total operation time of the method is no more than 5 minutes, or no more than 4 minutes, or no more than 1 minute.

According to an aspect of some embodiments of the invention, there is provided an electrochemical system or device comprising an electrochemical cell of some of the present embodiments and a means for contacting the gaseous sample with the carbon electrode.

According to some of any of the embodiments described herein, the means comprises an air sampler, for example, an air pump.

According to some of any of the embodiments described herein, the means comprises a breathing tube.

According to some of any of the embodiments described herein, the electrochemical system or device is a breathalyzer, as described herein.

According to some of any of the embodiments described herein, the electrochemical system or device is operable by contacting the gaseous sample with the carbon electrode to thereby provide a carbon electrode having the gaseous sample or a portion thereof absorbed thereto and integrating the electrode having the gaseous sample or a portion thereof absorbed thereto in said electrochemical cell.

According to some of any of the embodiments described herein, the electrochemical system or device operable by introducing the gaseous sample to said electrochemical cells.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B present simplified schematic presentations of exemplary electrochemical cells usable for detecting the presence, amount, and/or type of drugs in gaseous samples according to some of the present embodiments.

FIG. 2 presents a photograph showing an exemplary electrochemical cell assembly used in preliminary assays for electrochemical detecting cannabinoids in a solution, and exemplary cyclic voltammetry data obtained in these assays.

FIGS. 3A-J present cyclic voltammetry (CVA) data of CBD at a concentration range of 8-64 ppm (FIG. 3A) and current peaks at 1.065 volts as a function of CBD concentration (FIG. 3B, linear fitting correlation R=0.996); of THC at a concentration range of 6-48 ppm (FIG. 3C) and current peaks at 1.31 volts as a function of THC concentration (FIG. 3D, linear fitting correlation R=0.996); of THCA at a concentration range of 8-64 ppm (FIG. 3E) and current peaks at 1.42 volts as a function of THCA concentration (FIG. 3F, linear fitting correlation R=0.995); of CBN at a concentration range of 3-9 ppm (FIG. 3G) and current peaks at 1.48 volts as a function of CBN concentration (FIG. 3H, linear fitting correlation R=0.975); and of CBNA at a concentration range of 8-64 ppm (FIG. 3I) and current peaks at 1.42 volts as a function of CBNA concentration (FIG. 3J, linear fitting correlation R=0.988). Cyclic voltammetry was performed in an electrochemical cell with a carbon paper electrode (SPECTRACARB™ 2050A-1050, 0.35 cm²) working electrode with polyethylene insulation, platinum wire (0.4 cm²) counter electrode, Ag—AgCl (deposition) wire (0.4 cm²) reference electrode, and 50 mM tetrabutylammonium perchlorate in 70:30 water:acetonitrile as electrolyte, at the following cyclic voltammetry mode; E_{begin}=−0.5 V, Eᵥₑᵣₜₑₓ₁=1.4 V, Eᵥₑᵣₜₑₓ₂=−0.5 V, scan rate=0.1 V/sec, step=4 mV, number of scans=1.

FIGS. 4A-B present cyclic voltammetry (CVA) data of Meth at a concentration range of 4-32 ppm (FIG. 4A) and current peaks at 1.44 volts as a function of Meth concentration (FIG. 4B, linear fitting correlation R=0.998). Cyclic voltammetry was performed in an electrochemical cell with a carbon paper electrode (SPECTRACARB™ 2050A-1050, 0.35 cm²) working electrode with polyethylene insulation, platinum wire (0.4 cm²) counter electrode, Ag—AgCl (deposition) wire (0.4 cm²) reference electrode, and 50 mM tetrabutylammonium perchlorate in 70:30 water:acetonitrile solution as electrolyte, at the following cyclic voltammetry mode; E_{begin}=−0.5 V, Eᵥₑᵣₜₑₓ₁=1.4 V, Eᵥₑᵣₜₑₓ₂=−0.5 V, scan rate=0.1 V/sec, step=4 mV, number of scans=1.

FIGS. 5A-B present a photo of an exemplary, homemade, portable air sampler bearing a silicon tubing for air collection and for fixation of a carbon electrode according to some of the present embodiments during air pumping (FIG. 5A) and a schematic illustration of an exemplary design of a breath collection device containing a sensing carbon electrode according to some of the present embodiments (FIG. 5B).

FIG. 6 presents a scanning electron microscope (SEM) image of an exemplary modified micro-carbon-fibers electrode (0.18 mm thick, type SPECTRACARB™ 2050A-1050).

FIGS. 7A-H present cyclic voltammetry (CVA) data (FIGS. 7A, 7C, 7E and 7G) of CBD vapors collected from a 1 mg solid CBD source through a vacuum air sampler at room temperature for different periods of collection time, and graphs showing the respective collection efficiency as a function of the collection time (FIGS. 7B, 7D, 7F and 7H, respectively), as measured at a voltage of 0.95V, in an electrochemical cell assembly of a platinum wire (0.4 cm²) counter electrode, Ag—AgCl (deposition) wire (0.4 cm²) reference electrode, and 50 mM tetrabutylammonium perchlorate in 70:30 water:acetonitrile electrolyte solution, and varying working electrodes: carbon paper electrode (SPECTRACARB™ 2050A-1050, 0.35 cm²) (FIGS. 7A-B), carbon paper electrode modified with 5-fluorobenzene siloxane (FIGS. 7C-D), carbon paper electrode modified with dodecane siloxane (FIGS. 7E-F) and siloxane-(CF₂)₁₄ (FIG. 7G-H). E_{begin}=−0.5 V, Eᵥₑᵣₜₑₓ₁=1.4 V, Eᵥₑᵣₜₑₓ₂=−0.5 V, scan rate=0.1 V/sec, step=4 mV, number of scans=1.

FIG. 8 presents cyclic voltammetry (CVA) data of Meth vapors from a 2 mg sample (red) and marijuana vapors after burning a 1.7 mg sample (blue), compared to a room air sample (black). Working electrode was fluorosilane-modified carbon paper electrode (SPECTRACARB™ 2050A-1050, 0.35 cm²) with polyethylene insulation, counter electrode was platinum wire (0.4 cm²), reference electrode was Ag—AgCl (deposition) wire (0.4 cm²). The electrochemical background was 50 mM tetrabutylammonium perchlorate in 70:30 water:acetonitrile solution. E_{begin}=−0.5 V, Eᵥₑᵣₜₑₓ₁=1.4 V, Eᵥₑᵣₜₑₓ₂=−0.5 V, scan rate=0.1 V/sec, step=4 mV, number of scans=1.

FIG. 9 presents cyclic voltammetry (CVA) data of human breath samples collected for a time period of 30 seconds before and after smoking of cannabis, using an air collection device as depicted in FIG. 6 equipped with a fluorosilane-modified carbon paper electrode (SPECTRACARB™ 2050A-1050, 0.35 cm²) with polyethylene insulation, as a working electrode, platinum wire (0.4 cm²) as a counter electrode and Ag—AgCl wire (0.4 cm²) as a reference electrode (deposition). The electrochemical background was 50 mM tetrabutylammonium perchlorate in 70:30 water:acetonitrile. E_{begin}=−0.5 V, Eᵥₑᵣₜₑₓ₁=1.4 V, Eᵥₑᵣₜₑₓ₂=−0.5 V, scan rate=0.1 V/sec, step=4 mV, number of scans=1.

### DESCRIPTION OF SPECIFIC EMBODIMENTS OF THE INVENTION

The present invention, in some embodiments thereof, relates to electrochemical sensing and, more particularly, but not exclusively, to a system and method for electrochemically detecting a presence, amount and/or type of a drug in a gaseous sample.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

As discussed in the Background Section hereinabove, several electrochemical methods for detecting recreational drugs such as cannabinoids and amphetamines have been described in the literature. However, the task of real-time detection and monitoring of these compounds from a gaseous sample such as a human breath sample is still a great challenge.

In a search for a fast and efficient methodology for detecting electroactive drugs such as cannabinoids and amphetamines, the present inventors have conceived using a carbon electrode, preferably a carbon paper microelectrode, as the working electrode.

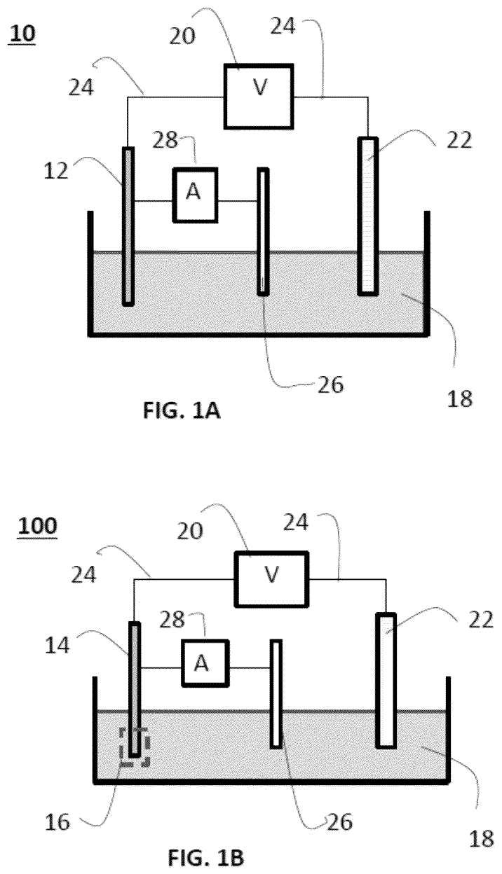

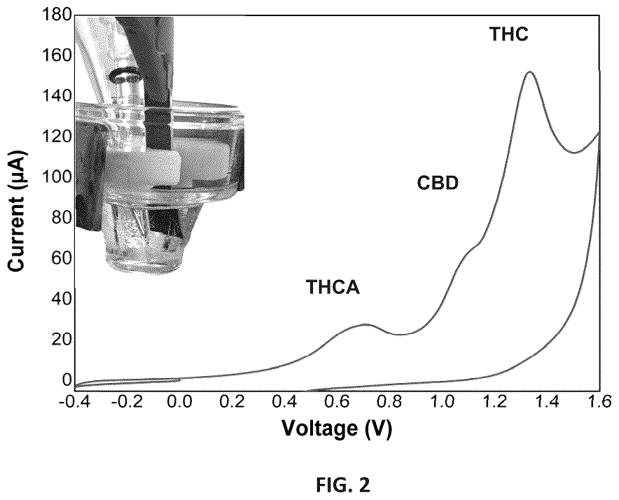

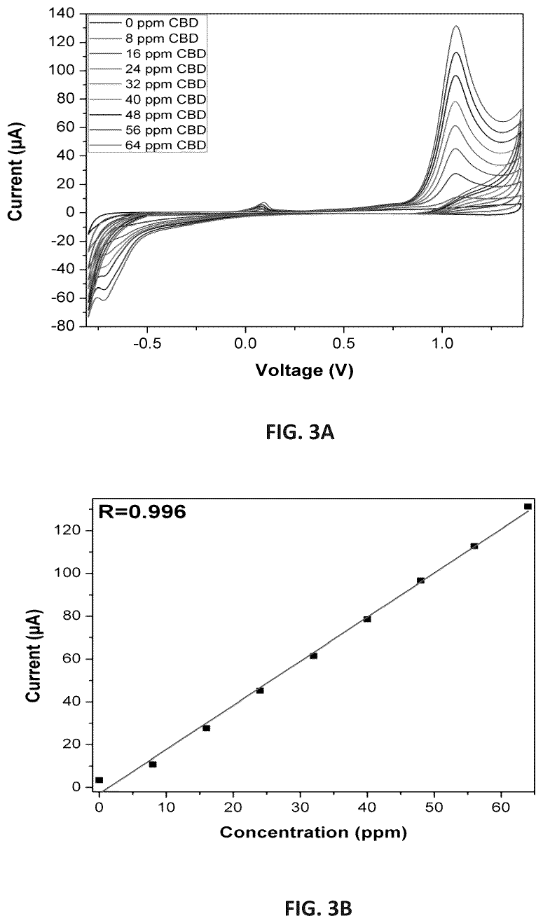

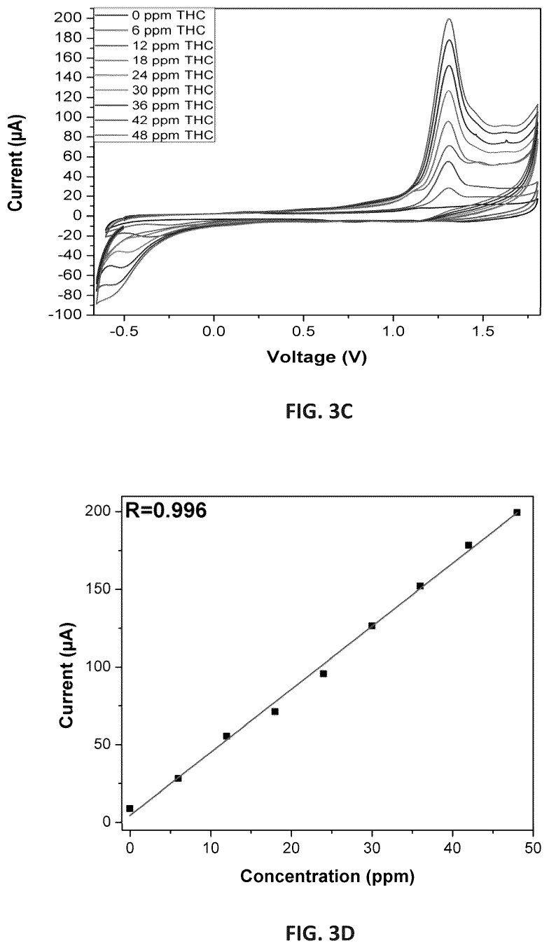

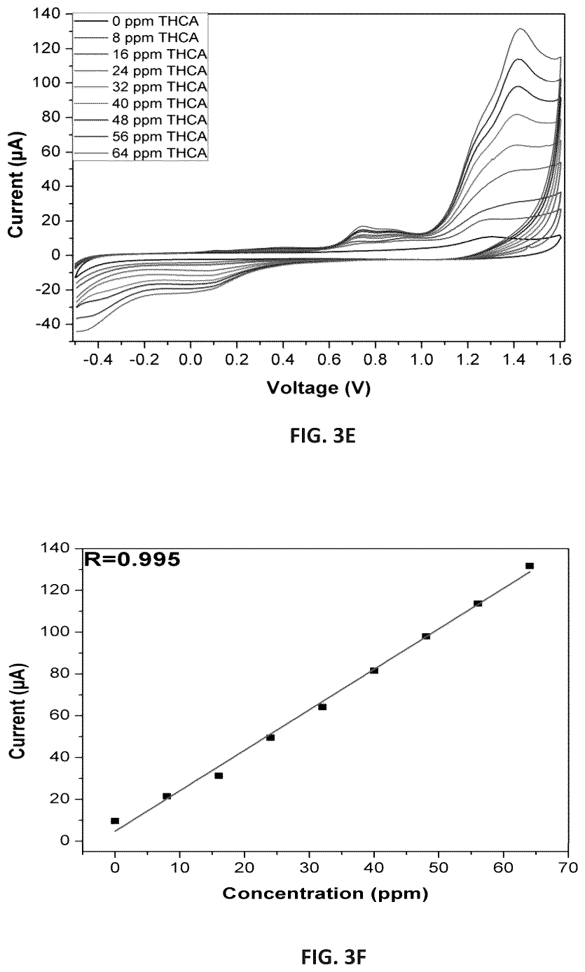

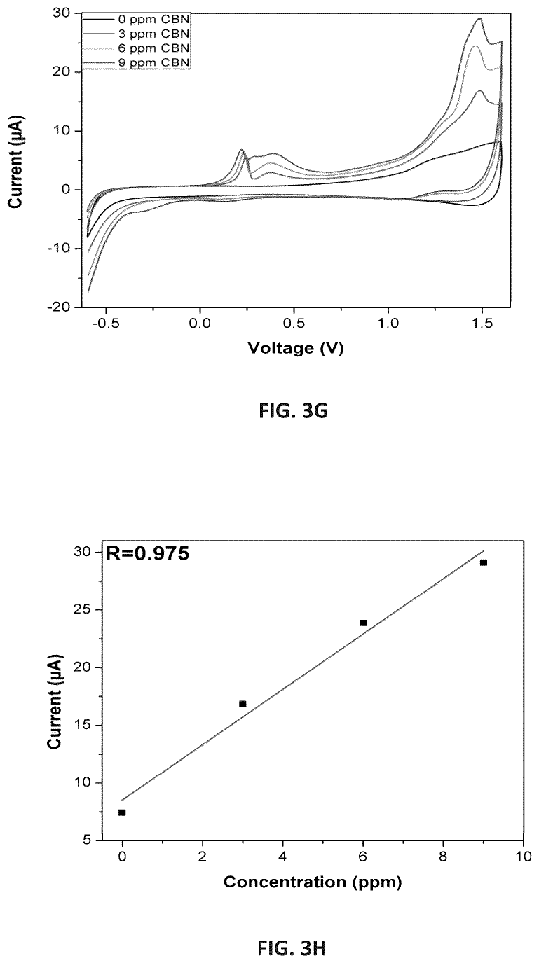

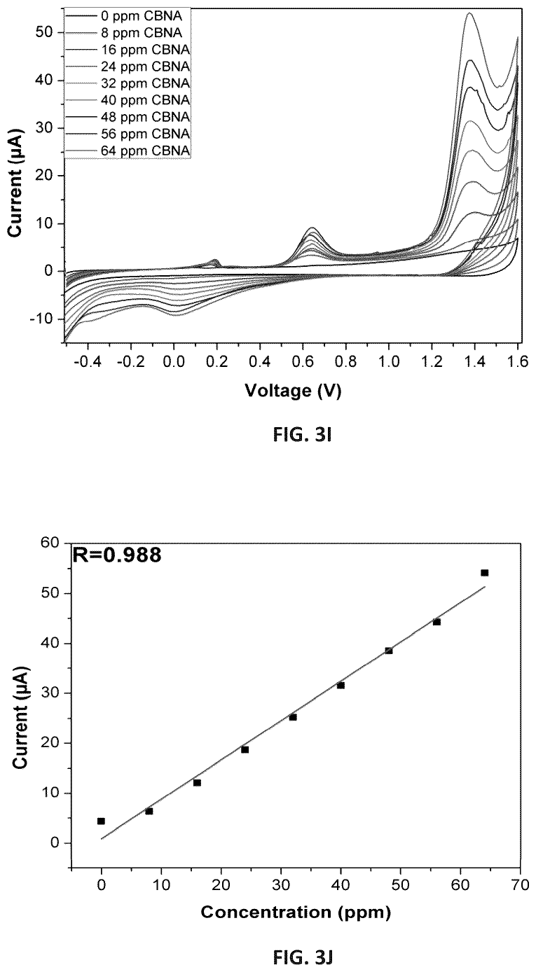

The present inventors have assembled an electrochemical cell as depicted, for example, in FIGS. 1A-B, for electrochemical cell 10 or electrochemical cell 100, respectively, having a carbon paper microelectrode 12, and have demonstrated an efficient detection of various cannabinoids in solution. See, e.g., FIGS. 2, 3A-J, and 4A-B.

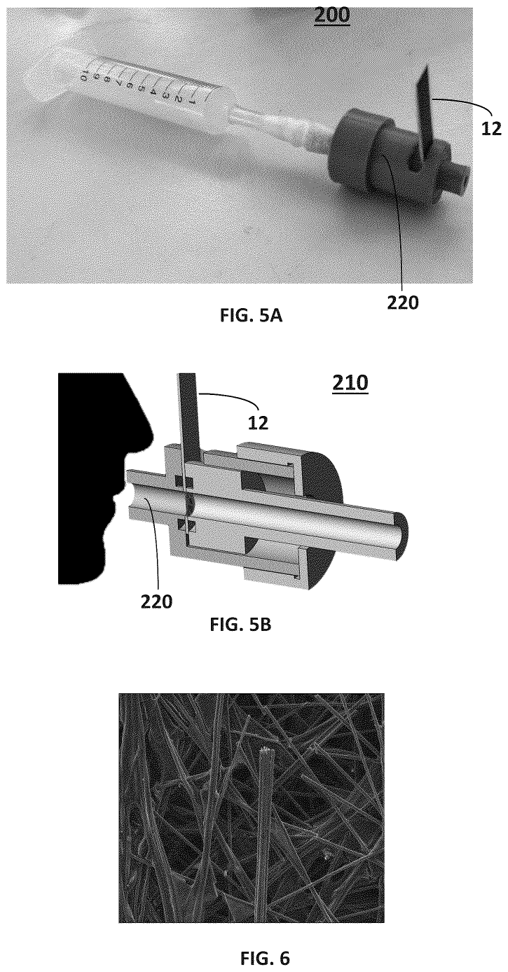

In order to explore the possibility of such an electrode to detect electroactive drugs in a gaseous sample, the present inventors have assembled an air-sampling device a schematic illustration of an exemplary design of a breath collection device 200 containing a sensing carbon electrode 12 according to the present embodiments, as depicted, e.g., in FIG. 5A.

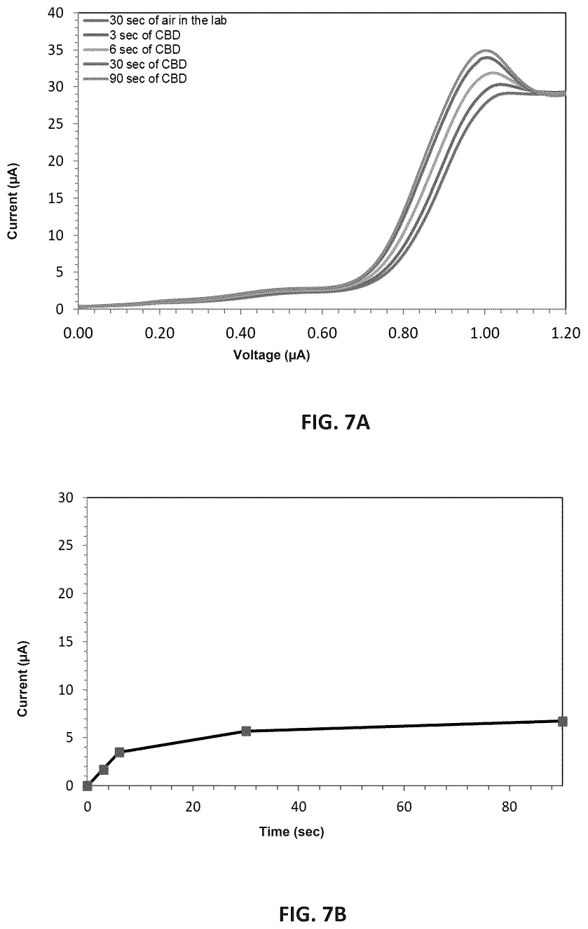

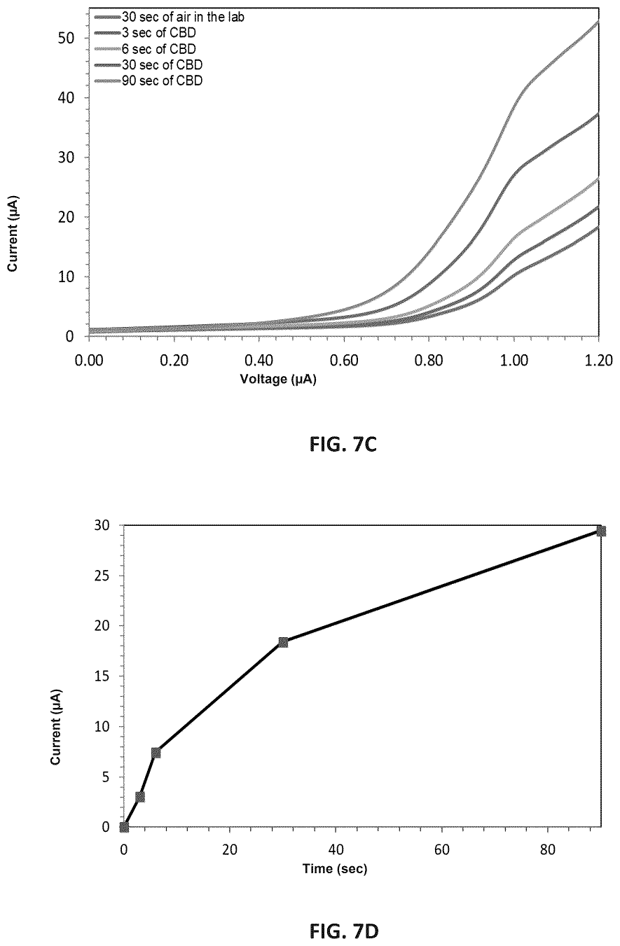

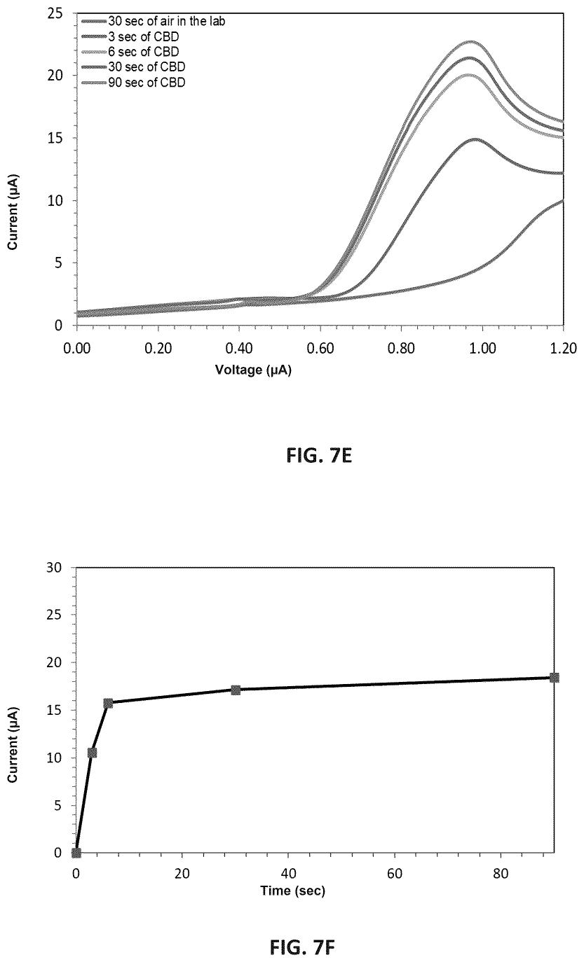

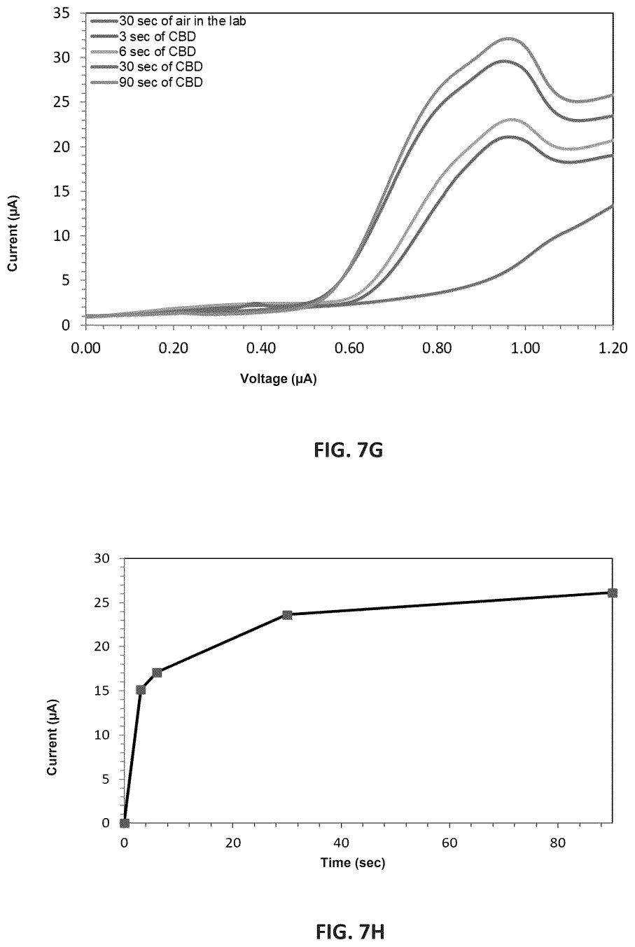

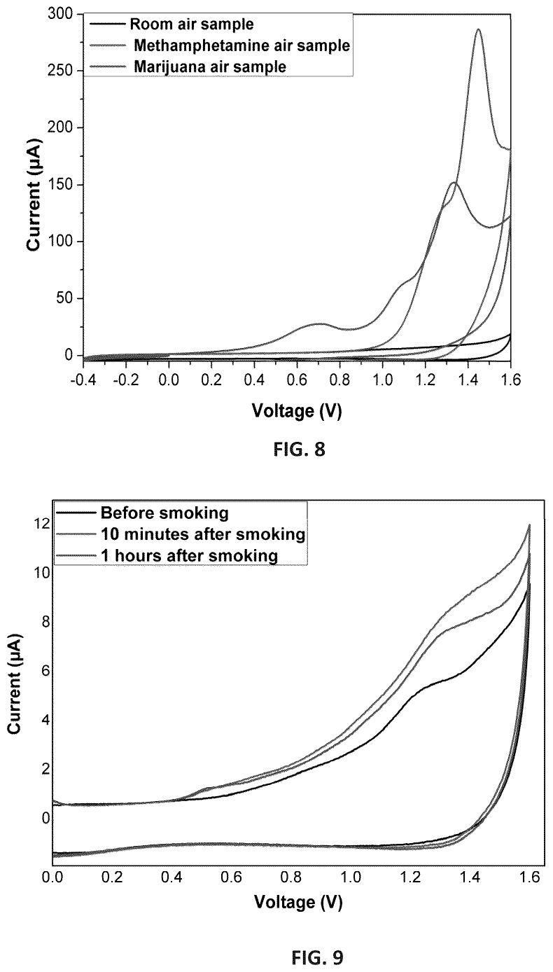

Since the vapor pressure of electroactive drugs such as cannabinoids is typically low, the present inventors have conceived modifying the electrode's surface in order to enhance the amount of the drug that is absorbed thereto, while maintaining an efficient electron transfer to allow electrochemical reaction and detection. The present inventors have tested several modifications and have shown that an efficient detection is performed upon attaching to the electrode a hydrophobic moiety that bears a polar substituent, see, e.g., FIGS. 7A-H. An efficient detection of gaseous samples using a modified carbon paper electrode (such as shown, for example, in electrochemical cell 100 in FIG. 1i), has been demonstrated, as shown e.g., in FIGS. 8 and 9, indicating the efficient and sensitive detection of drugs in gaseous samples such as a human breath sample.

Embodiments of the present invention therefore relate to a sensing system or device, which is configured for detecting electroactive drugs in a gaseous sample, such as a human breath sample, and to method of detecting a presence, amount, and/or type of an electroactive (electrochemically detectable) drug in a gaseous sample such as a human breath sample.

Embodiments of the present invention further relate to a method of detecting a presence, amount and/or type of an electroactive (electrochemically detectable) drug such as cannabinoids and amphetamines in a gaseous sample. The method is generally effected by contacting the sample with a carbon electrode; applying potential to the electrode; and measuring an electrochemical parameter of the carbon electrode, wherein the electrochemical parameter is indicative of a presence and/or amount and/or type of the electroactive drug in the sample.

Embodiments of the present invention further relate to modified carbon electrodes usable for detecting an electroactive drug, to electrochemical cells comprising a carbon electrode as described herein, and to devices or systems, which comprise such electrochemical cells, which are configured for detecting electroactive drugs in a gaseous sample.

According to an aspect of some embodiments of the invention, there is provided a method of detecting a presence and/or amount and/or type of a drug in a sample.

In some of any of the embodiments described herein, the method as described herein is a method of electrochemical detection of a presence and/or amount and/or type of an electrochemically detectable (electroactive) drug in a (e.g., gaseous) sample.

Herein throughout, the terms “detection”, “detecting” and grammatical diversions thereof, and the terms “sensing”, are used interchangeably, and describe determining a presence and/or amount and/or type of a substance in a sample, herein of a drug is a sample such as a gaseous sample.

The method according to the present embodiments is generally effected by contacting a sample with a carbon electrode as described herein in any of the respective embodiments and any combination thereof, which is also referred to herein interchangeably as “sensing electrode” or simply as “electrode”, and applying potential to the carbon electrode.

In some of any of the embodiments described herein, the method further comprises measuring an electrochemical parameter or a change in an electrochemical parameter of the carbon electrode (compared, for example, to the same parameter when not contacted with the sample, and when contacted with a sample devoid of a drug), wherein the electrochemical parameter or the change in the electrochemical parameter is indicative of the presence and/or amount and/or type of the drug in the sample, as described in further detail hereinunder.

A method as described herein is for detecting a presence of a drug in a sample. By “drug” it is meant a substance that can alter a physiological function in a physiological system (e.g., a body of a subject), and encompasses pharmaceutical drugs, which are used to treat, prevent and/or diagnose a medical condition (a disease or disorder) in a subject, and recreational drugs, which exhibit a psychotropic effect, and can alter mood, perception and/or consciousness in a subject.

Herein throughout, the term “drug” encompasses the various states or phases in which the drug may be found in the (e.g., gaseous) sample, including an intact drug, a heated drug, and/or a consumed drug. This includes scenarios where the drug is in its original form (an intact drug), where the drug is subjected to thermal processes that may alter its physical and/or chemical properties (e.g., a heated drug), and/or where a drug is consumed (e.g., through smoking; a smoked drug, or otherwise through oral ingestion or through any other consumption mode), which may involve decomposition and/or combustion products of the drug in the (e.g., gaseous) sample.

According to some of the present embodiments, drugs that can be detected using a method as described herein include electrochemically detectable drugs.

Herein throughout, the phrase “electrochemically detectable drug” is also referred to herein interchangeably as “electroactive drug”, and describes a drug that is capable of undergoing a redox reaction upon potential application. This term encompasses also a drug that is capable of releasing or generating (e.g., upon consumption) a compound that is capable of undergoing a redox reaction upon potential application. This term encompasses also a drug that is prepared from, or which its preparation involves the production of (e.g., as an intermediate or side product), a compound that is capable of undergoing a redox reaction upon potential application.

According to some of any of the embodiments described herein, the phrase “electrochemically detectable drug” or “electroactive drug” describes an intact drug that is capable of undergoing a redox reaction upon potential application.

As used herein, the phrase “capable of undergoing a redox reaction upon potential application” describes a compound which can either accept or donate electrons, leading to a change in its oxidation state when subjected to an applied potential and to the generation or consumption of electrodes as a result of potential application.

Herein throughout, an “electrochemically detectable drug” which is also referred to herein as an “electroactive drug”, is also referred to herein simply as a “drug”.

A method as described herein, by allowing fast and efficient detection of drugs in a sample, including a non-processed sample, is useful for detecting illegal or otherwise recreational drugs.

According to some of any of the embodiments described herein, the electroactive drug is a recreational drug.

As used herein and in the art, the phrase “recreational drug” describes a substance that may alter the state of consciousness or the perception of a subject administered therewith, typically a human subject. This phrase encompasses also substances that are intended for use as a pharmaceutical drug, but which may affect the subject's consciousness or perception, as the intended effect or as a side effect.

Non-limiting examples for recreational drugs include alcohol, nicotine, caffeine, hallucinogenic and dissociative drugs (non-limiting examples include psilocybin mushrooms, PCP (phenylcyclohexyl piperidine), DMT (dimethyltryptamine), mescaline (peyote; 3,4,5-trimethoxyphenethylamine), 2C (e.g., 2C-B; 2,5-dimethoxy-4-bromophenethylamine), DXM (dextromethorphan), ayahuasca, LSD (lysergic acid diethylamide), Salvia (Salvia divinorum), DOB (2,5-dimethoxy-4-bromoamphetamine), ketamine), heroin, khat, kratom, GHB and/or its analogs, ecstasy (3,4,-methylenedioxymethamphetamine, MDMA), cannabis and hashish (e.g., THC) and cannabinoids in general, cocaine, methamphetamines, synthetic cannabinoids (non-limiting examples include Spice, K2); and pharmaceuticals which are commonly abused as drugs, such as opioids (non-limiting examples include morphine, methadone, fentanyl, oxycodone, hydrocodone, and codeine), sedatives (e.g., barbiturates; benzodiazepines (such as diazepam, lorazepam, and temazepam); zolpidem (available as, e.g., AMBIEN®)), cough suppressants, dissociative anesthetics (non-limiting examples include esketamine and dextromethorphan (DXM)), stimulants (non-limiting examples include amphetamine, methylphenidate, and methamphetamine), anabolic steroids (non-limiting examples include testosterone, nandrolone, and stanozolol), gabapentinoids (non-limiting examples include gabapentin and pregabalin), phenobarbital, inhalants (non-limiting examples include nitrous oxide, amyl nitrite), designer drugs (non-limiting examples include synthetic cannabinoids, synthetic cathinones (bath salts)), and antidepressants (non-limiting examples include fluoxetine, sertraline).

According to some of any of the embodiments described herein, the electroactive drug is a recreational electroactive drug, such as, but not limited to, opioids, amphetamines, cannabinoids, LSD, and like drugs.

Herein, a drug is also referred to interchangeably as an analyte.

According to some of any of the embodiments described herein, the electroactive drug is lipophilic, as defined herein.

As used herein and in the art, the term “lipophilic” describes a hydrophobic substance having a tendency to dissolve in and/or associate to fatty or fat-like solvents.

As used herein throughout, the terms “lipophilic” and “hydrophobic” describe a physical property of a material or a portion of a material (e.g., a chemical group in a compound) which does not form bond(s) with water and tend to associate with non-polar substances. Hydrophobic and lipophilic materials dissolve more readily in oil than in water.

Hydrophobicity is commonly measured by its distribution behavior in a biphasic system: either liquid-liquid (e.g., partition coefficient in 1-octanol/water, as described herein) or solid-liquid (retention on reversed-phase high-performance liquid chromatography (RP-HPLC) or thin-layer chromatography (TLC) system).

Hydrophilic, amphiphilic, lipophilic, and hydrophobic substances can be determined by the partition coefficient thereof. A partition coefficient is the ratio of concentrations of a compound in the two phases of a mixture of two immiscible liquids at equilibrium, typically at room temperature. Normally, one of the solvents chosen is water while the second is hydrophobic such as n-octanol. The logarithm of the ratio of the concentrations of the un-ionized solute in the solvents is called Log P.

Hydrophobic substances are characterized by Log P higher than 1; hydrophilic substances are characterized by Log P lower than 1; and amphiphilic substances are characterized by Log P of about 1 (e.g., 0.8-1.2).

Hydrophobicity can alternatively, or in addition, be determined by a lipophilicity/hydrophilicity balance (HLB), according to the Davies method, lower than 3.

In some of any of the embodiments described herein, the drug is a lipophilic recreational drug. Non-limiting examples for lipophilic recreational drugs include cannabinoids, amphetamines, opioids (e.g., fentanyl, alfentanil, sufentanil, remifentanil), benzodiazepines, and certain barbiturates (depending on their side chain substitutions).

According to some of any of the embodiments described herein, the electroactive drug is a cannabinoid, including cannabinoids that cause a psychotropic effect and those that do not cause such an effect.

As used herein and in the art, the term “cannabinoid” describes a chemical substance that act on cannabinoid receptors in cells, and/or have similar effects to cannabinoid substances produced by a Cannabis plant. Cannabinoid are naturally found in the Cannabis plant. Herein, this term encompasses natural cannabinoids derived from a cannabis plant, natural cannabinoids extracted from a cannabis plant, including compositions comprising such extracts, and synthetically produced cannabinoids or analogs thereof (which provide the same effect). Non-limiting examples of natural cannabinoids include tetrahydrocannabinol (THC), tetrahydrocannabinolic acid (THCA), cannabidiol (CBD), cannabinol (CBN), cannabigerol (CBG), cannabichromene (CBC), tetrahydrocannabivarin (THCV), cannabidivarin (CBDV), and cannabidiolic acid (CBDA). Non-limiting examples of synthetic cannabinoids include Spice, K2, JWH-018, JWH-073, HU-210, AM-2201, CP 47,497, and UR-144.

According to some of any of the embodiments described herein, the electroactive drug is an amphetamine.

Amphetamines are a class of central nervous system stimulants that increase the levels of certain neurotransmitters in the brain, primarily dopamine and norepinephrine. Commonly used amphetamines include: Amphetamine (Benzedrine); Dextroamphetamine (Dexedrine) and Methamphetamine.

In some of any of the embodiments described herein, the amphetamine is methamphetamine.

According to some of any of the embodiment described herein, the drug, or a portion thereof, is in a gaseous state.

By “gaseous state” it is meant that at least a portion of the drug is in a form of vapors. Thus, for example, the drug can be a liquid or a solid at room temperature, yet, it is volatile to some extent, such that at least a portion thereof is in a gaseous state at a temperature at which the gaseous sample is being collected (e.g., room temperature, a physiological temperature).

Herein throughout, by “room temperature” it is meant an ambient temperature, typically ranging from about 15 to about 30, or from about 15 to about 25, or from about 20 to about 30, or from about 20 to about 25, ° C.

As known in the art, each compound is characterized by a phase diagram that illustrates its state (solid, liquid, or gas) under different conditions of temperature and pressure. A drug which is not gaseous at the conditions at which the sample is contacted with the electrode, e.g., at ambient conditions (e.g., the is solid and/or fluid as described herein) may be at least partially in gaseous state (e.g., is characterized by a vapor pressure of at least 0.1 kPa at room temperature) at these conditions or upon heating and/or reducing pressure of a sample comprising the drug.

According to some of any of the embodiments described herein, the electroactive drug is a volatile drug.

Herein and in the art, the term “volatile” describes a substance that easily evaporates under specified conditions (e.g., at standard temperature and pressure (STP) conditions). Volatility is measured by a substance's vapor pressure under specified conditions (e.g., room temperature) when placed in a closed container.

According to some of any of the embodiments described herein, the electroactive drug is volatile, such that it features a vapor pressure of at least 0.1 kPa, or at least 1 kPa, or at least 10 kPa, or at least 100 kPa, or at least 1 MPa, or even higher, at room temperature.

According to some of any of the embodiments described herein, the electroactive drug is non-volatile, and features a vapor pressure of no more than 10 Pa, or no more than 1 Pa, or no more than 0.1 Pa, or no more than 0.01 Pa, or even lower, at room temperature.

According to some of any of the embodiments described herein, the drug is in a solid state or liquid state at the temperature at which the sample is contacted with the electrode (e.g., room temperature), yet, a portion of the drug is in a gaseous state, and this portion is detectable by a method as described herein.

In some of any of the embodiments described herein, a detectable concentration of the drug (in a gaseous state) in the sample as described herein in any of the respective embodiments is no more than 1000, or no more than 500, or no more than 250, or no more than 100, or no more than 50, or no more than 20, or no more than 15, or no more than 10, or even no more than 5, ng/liter, of the drug (in a gaseous state), including any intermediate values and subranges therebetween.

According to some of any of the embodiments described herein, the gaseous sample comprises no more than 10 ng/liter of the drug (in a gaseous state).

An actual concentration of the drug (in its gaseous state) in the sample, can be higher, and can range, for example, from 5 mg/liter to 10 grams/liter, or from 10 mg/liter to 10 grams/liter, or from 20 mg/liter to 10 grams/liter, or from 50 mg/liter to 10 grams/liter, or from 100 mg/liter to 10 grams/liter, or from 1 gram/liter to 10 grams/liter, or from 5 mg/liter to 5 grams/liter, or from 10 mg/liter to 5 grams/liter, or from 20 mg/liter to 5 grams/liter, or from 50 mg/liter to 5 grams/liter, or from 100 mg/liter to 5 grams/liter, or from 1 gram/liter to 5 grams/liter, or from 5 mg/liter to 1 gram/liter, or from 10 mg/liter to 1 gram/liter, or from 20 mg/liter to 1 gram/liter, or from 50 mg/liter to 1 gram/liter, or from 100 mg/liter to 1 gram/liter, including any intermediate values and subranges therebetween.

In some of any of the embodiments described herein, the sample is a fluid sample at the temperature it is contacted with the electrode, and can be a liquid sample or a gaseous sample. In some of any of the embodiments described herein, the drug is in a fluid state (e.g., is in a liquid state or a gaseous state) at the temperature the sample is contacted with the electrode.

The term “fluid” is defined as a substance that tends to flow and to conform to the outline of its container. Typical fluids include liquids and gasses, but may also include free flowing solid particles.

In some of any of the embodiments described herein, the sample is a solid sample at the temperature at which it is contacted with the electrode.

In some of any of the embodiments described herein, when the sample is a solid sample or a liquid sample as described herein, at the temperature at which it is contacted with the electrode, it is characterized by a vapor pressure of at least 0.1 kPa, at this temperature.

Alternatively, the sample is subjected to heating and/or reducing pressure, so as to feature a vapor pressure of at least 0.1 kPa.

In some of any of the embodiments described herein, the drug, or at least a portion of the drug in a sample, is in a gaseous state upon heating and/or reducing pressure of a sample containing same.

According to some of any of the embodiments described herein, the electroactive drug is a volatile recreational drug.

Non-limiting examples of volatile recreational drugs include psilocybin mushrooms, ayahuasca, heroin, khat, kratom, GHB, GHB analogs, MDMA, cannabis and hashish, synthetic cannabinoids, opioids, benzodiazepines, cocaine, methamphetamines, inhalants, ketamine, PCP, Salvia divinorum, DMT, DXM, 2C-B.

According to some of any of the embodiments described herein, the electroactive drug is a non-volatile recreational drug, yet, it generates volatile products, side products, and/or by-products when consumed. For example, a non-volatile drug can generate volatile products upon combustion (e.g., upon smoking the drug), and these volatile products are in a gaseous state at the temperature at which the sample is contacted with the electrode.

According to the present embodiments, the sample, or at least a portion thereof, is gaseous at the temperature at which it is contacted with the electrode, that is, the method is effected by contacting a gaseous sample or a gaseous portion of the sample with an electrode as described herein in any of the respective embodiments and any combination thereof.

The sample encompasses samples suspected as containing a drug, such that the system, device and method as described herein in any of the respective embodiments is utilized for determining a presence, and optionally an amount (a concentration or a level) of a drug in the sample, and, optionally and preferably, an identity (e.g., the type, e.g., a chemical composition) of the drug. Optionally, the gaseous sample is known to contain a drug and the method, device and system described herein in any of the respective embodiments is utilized for determining an amount (level) and/or identity (e.g., the type, e.g., a chemical composition) of the drug as described herein in any of the respective embodiments.

According to some of any of the embodiments described herein, the gaseous sample is an air sample.

According to some of any of the embodiments described herein, the air sample is of an environment of the drug or of products generated upon its consumption as described herein, or of products generated or used during the production of the drug.

For example, the air sample can be a sample of an environment where a drug was consumed, in cases where the drug was consumed by smoking or combustion. For example, the air sample can be a sample of an environment where a drug was prepared. For example, the air sample can be a sample of an environment where a drug was stored or transferred. For example, the air sample can be a bodily air sample, for example, a breath sample of a subject suspected as consuming the drug.

A sample as described herein in any of the respective embodiments can be used as is, without being further processed, such that a sample such as an air sample is contacted with the carbon electrode once collected. Alternatively, the sample is treated so as to increase its vapor pressure, to thereby generate or increase a gaseous portion of the sample and/or of a drug comprised in the sample. Further alternatively, a sample is treated by subjecting it to conditions at which electroactive substances that are derived from a drug are generated. For example, a sample (which can be solid or liquid or gaseous) is heated, or subjected to combustion, so as to generate decomposition products of the drug which are electroactive.

In some of any of the embodiments described herein, the method comprises collecting the gaseous sample or a gaseous portion of the sample, e.g., using a collecting means. Non-limiting examples for collecting means include pumps such as air pumps, vials, syringes, gas sampling bags (such as those obtainable from, e.g., Tedlar®, Teflon®, Mylar®), stainless steel canisters, diffusion tubes, gas sampling pumps, and sorbent tubes. Exemplary means of collecting a gaseous sample are described in further detail hereinbelow. A collected sample can be contacted with a carbon electrode immediately after collection, or otherwise can stored and contacted with the carbon electrode later (e.g., after 1 hour, a few hours, or a few days), optionally after being treated as described herein. In some of any of the embodiments described herein, a sample is collected and contacted with the electrode simultaneously, such that, for example, the sample is collected by means that comprises the electrode and is immediately contacted with the carbon electrode. Exemplary means of collecting a gaseous sample are described in further detail hereinbelow.

In some of any of the embodiments described herein, the sample is a gaseous sample of a breath of a subject. The gaseous sample may be collected from a subject that is known or is suspected to consume a drug. According to some of any of the embodiments described herein, the gaseous sample is a breath sample. In some such embodiments, the gaseous sample is a breath sample of a subject. In some such embodiments, the subject is a human subject suspected as consuming the drug. According to embodiments where the sample is a gaseous sample of a breath of a subject as described herein, the sample is contacted with the carbon electrode by having the subject breathing onto the carbon electrode, or onto or into a device or system that comprises the carbon electrode and the carbon electrode is in fluid communication with the collected sample, as exemplified in further detail hereinbelow.

The gaseous sample (e.g., an air sample) may alternatively be collected from different locations, preferably locations that are suspected or known to involve a drug-consuming and/or drug-manufacturing activity. Exemplary such locations include, without limitations, indoor air samples (e.g., homes, offices, hospitals and caregiving centers, vehicles, gyms, public bathrooms, factories, clandestine laboratories, pharmaceutical manufacturing plants, chemical processing facilities, storage warehouses, distribution centers, social venues such as bars and parties) and outdoor air samples (e.g., from urban, rural, and remote environments; public parks, parking lots, beaches, streets, alleys, recreational areas, industrial parks, abandoned properties, areas near chemical disposal sites, social public events such as concerts and festivals). In some of these embodiments, the gaseous sample can be collected and used as described herein, for example, by means of an air pump or an air sampler. The air pump or air sampler can comprise the carbon electrode. Alternatively, the sample is collected, stored, and then contacted with the electrode, as described herein in any of the respective embodiments.

In some of any of the embodiments described herein, the method comprises collecting the non-gaseous sample (e.g., a solid sample or a fluid sample as described herein), and treating the non-gaseous sample to generate a gaseous sample. Means for collecting a non-gaseous sample are well known in the art, and include, for example, vials, scoops, syringes, containers, and the like. In some of any of the embodiments described herein, treating the non-gaseous sample to generate a gaseous sample comprise heating and/or reducing pressure of a sample comprising the drug.

In some of any of the embodiments described herein, a sample can comprise one or more drugs, and at least one of the drugs is an electroactive drug as described herein in any of the respective embodiments and any combination thereof.

In some of any of the embodiments described herein, a sample comprises more than one drug, each is independently an electroactive drug as described herein in any of the respective embodiments and in any combination thereof, and each of the electroactive drugs has a different value of the electrochemical parameter as described herein in any of the respective embodiments. That is, the two or more drugs are distinguishable at least by the electrochemical parameter.

According to an aspect of some embodiments of the invention, there is provided a carbon electrode capable of detecting a presence and/or amount and/or type of a drug (an electrochemically detectable drug as described herein in any of the respective embodiments, in a sample such as a gaseous sample, as described herein in any of the respective embodiments and any combination thereof.

According to some of any of the embodiments described herein, a carbon electrode as described herein is usable for electrochemical detection of a drug in a (e.g., gaseous) sample, as these are described herein in any of the respective embodiments.

In some of any of the embodiments described herein, the carbon electrode as described herein is usable for electrochemically determining a presence, type and/or level of an electroactive drug in a gaseous sample, as these are described herein in any of the respective embodiments.

In some of any of the embodiments described herein, the carbon electrode as described herein is usable for electrochemically determining a type and/or a presence and/or level of an electroactive drug in a gaseous sample, as these are described herein in any of the respective embodiments.

According to some embodiments of the present invention, the carbon electrode features at least one nanoscale or microscale dimension.

By “microscale dimension” it is meant that at least one dimension of the electrode is lower than 1 mm, or ranges from 0.1 micron to 900 microns.

By “nanoscale dimension” it is meant that at least one dimension of the electrode is lower than 1 micron, or ranges from 0.1 nanometer to 900 nanometers.

The nanoscale or microscale dimension depends on the shape of the electrode. If an electrode is generally shaped as a cylinder, the at least one dimension can be one or both of a length and a diameter of the electrode. If the electrode is generally shaped as a rectangular, the at least one dimension can be one or more of a length and a width of the electrode.

Electrodes featuring one or more microscale or nanoscale dimension are also referred to herein and in the art as microelectrodes.

Carbon electrodes or microelectrodes can be made of glassy carbon, screen-printed carbon, carbon films, carbon fibers, carbon paste, carbon nanotubes and others.

According to some of any of the embodiments described herein, the carbon electrode is a carbon fiber electrode.

A carbon fiber (CF) electrode is an electrode that comprises elementary carbon (e.g., graphite) shaped as a fibrous structure (e.g., a filament). Generally, but not necessarily, a CF electrode features a microscale or even nanoscale diameter or thickness (width), typically, but not limited to, in a range of from 1 to 500 microns, or from 5 to 200 microns, or 5 to 100 microns, or 5 to 50 microns or 5 to 20 microns. Generally, but not necessarily, a CF electrode features a length (height) of from about 100 microns to about 50 mm, or from about 100 microns to about 1 mm, or from about 100 microns to about 800 microns, including any intermediate values and subranges therebetween. CF electrode featuring at least one dimension in the microscale or nanoscale range is a CF microelectrode.

According to some of any of the embodiments described herein, the carbon electrode is a carbon fiber microelectrode.

In some embodiments the CF microelectrode further comprises a mechanical support enveloping or surrounding at least a portion of the electrode, leaving a protruding tip of e.g., from 10 to 100 microns, of unsupported, exposed portion of the electrode.

The CF microelectrode can be a single-barrel or a multi-barrel electrode.

The CF microelectrodes can be carbon fabric electrodes or carbon paper electrodes. The carbon fabric electrodes can be made of woven or non-woven carbon filaments or bundles of filaments.

Any commercially available CF microelectrode can serve as a raw material for providing a CF microelectrode according to the present embodiments.

According to some of any of the embodiments described herein, the carbon electrode is a carbon paper microelectrode (also referred to herein interchangeably as a “micro carbon paper electrode”).

In some of any of the embodiments described herein, the carbon electrode (e.g., as described herein in any of the respective embodiments) is characterized by a surface area of at least 10-50 cm² per geometrical cm², including any intermediate value and subranges there between.

According to some of any of the embodiments described herein, the carbon electrode is a gas-permeable electrode.

As used herein and in the art, the phrase “gas-permeable electrode” describes an electrode that allows gases to pass through its structure while maintaining its electrical conductivity. Gas permeable electrodes allow sensing of gaseous samples (e.g., air) and/or analytes while circumventing the need to introduce the sample via a dedicated gas inlet.

By “gas-permeable” it is meant that the electrode is characterized by air permeability through plane higher than 0.3 cfm/ft² through 0.25 mm, when measured according to standard assays such as ASTM 737-96, ISO 5636, ISO 4638, ISO 9237, and TAPPI T460.

Exemplary commercially available gas-permeable carbon fiber electrodes that are usable in the context of the present embodiments include, but are not limited to, plain carbon cloth such as, for example, electrodes marketed as ELAT—Hydrophilic Plain Cloth®, 1071 HCB plain carbon cloth, Zoltek™ Panex 30.

Exemplary commercially available gas-permeable carbon paper electrodes that are usable in the context of the present embodiments include, but are not limited to, electrodes marketed by Freudenberg FCCT, such as Freudenberg H23, electrodes of the Spectracarb™ family, Sigracet 39 AA, electrodes marketed under the trade name AvCarb® (e.g., AvCarb P75), and similar gas-permeable carbon paper electrodes.

Any commercially available gas-permeable carbon electrode (e.g., as described herein in any of the respective embodiments) can serve as a carbon electrode according to any of the embodiments described herein, either as is or upon being treated or modified as described herein.

According to some of any of the embodiments described herein, the carbon electrode is pre-treated before being used in a method as described herein and/or before being assembled in an electrochemical cell or half-cell, as described herein. For example, the carbon electrode can be washed with, or immersed in, an organic solvent, and optionally dried (e.g., air-dried) thereafter.

According to some of any of the embodiments described herein, the carbon electrode is a chemically-modified electrode.

According to some of the present embodiments, there is a provides a modified carbon electrode, preferably a modified gas-permeable carbon electrode as described herein in any of the respective embodiments, which comprises one or more functional moieties as described herein in any of the respective embodiments, attached (e.g., covalently) to at least a portion of the surface of the electrode.

According to some of any of the embodiments described herein, the carbon electrode (e.g., a gas-permeable carbon electrode) features one or more functional moiety/moieties. In some of any of the embodiments described herein, a functional moiety is attached (covalently) to at least a portion of the surface of the carbon electrode (e.g., to an exposed portion of the surface of the carbon electrode as described herein in any of the respective embodiments). In some of any of the embodiments described herein, a functional moiety is covalently attached to at least a portion of the surface of the carbon electrode as described herein.

In some of any of the embodiments described herein, the carbon electrode features a plurality of functional moieties as described herein in any of the respective embodiments, and the plurality of functional moieties are attached to at least a portion of the surface of the carbon electrode. In some of these embodiments, at least a portion, and preferably most or all, of the functional moieties, are covalently attached to at least a portion of the surface of the carbon electrode.

In some of any of the embodiments described herein, an average density of the functional moieties on the carbon electrode surface is at least 10³, or at least 10⁵, or at least 10⁸, or at least 10¹⁰ moieties per cm², or at least 10¹¹ moieties per cm², and can be, for example, from about 10³ to about 10¹³, or from about 10¹⁰ to about 10¹³, functional moieties per cm² of the electrode's surface.

Any commercially available gas-permeable carbon electrode (e.g., as described herein in any of the respective embodiments) can serve as a raw material for providing a chemically-modified carbon electrode according to some of any of the present embodiments, upon generating on at least a part of its surface a functional moiety as described herein. Alternatively, commercially available chemically-modified carbon electrodes are used.

According to some of any of the embodiments described herein, the functional moiety is selected as capable of interacting with the electroactive drug, and/or as capable of transferring electrons through the electrode (e.g., between the electroactive drug and the carbon electrode as described herein). According to some of any of the embodiments described herein, a functional moiety that is or comprises a lipophilic moiety (a hydrophobic moiety) is usable, particularly in cases where the electroactive drug is lipophilic, as described herein, for example, is a cannabinoid.

According to some of any of the embodiments described herein, the functional moiety is attached to at least a portion of the surface of the electrode (e.g., an exposed portion of the surface of the carbon electrode as described herein in any of the respective embodiments). This allows detecting traces or low concentrations of the electroactive drug as defined herein.

In some of any of the embodiments described herein, the functional moiety is selected capable of transferring electrons through the electrode. This may be effected, e.g., by means of a functional moiety that features polarity and/or aromaticity.

According to some of any of the embodiments described herein, the functional moiety is selected capable of associating with a target drug (the drug to be detected).

By “associating with” and grammatical diversions thereof it is meant herein throughout interacting with the drug via one or more chemical interactions, for example, hydrophobic interactions, electrostatic interactions, Van der Waals interactions, hydrogen bonding, aromatic interactions, and more.

Without being bound by any particular theory, it is assumed that a functional that is capable of associating with a target drug increases the amount of drug that contacts the carbon electrode and is absorbed to the electrode (e.g., is physically associated with the electrode's fibers), and thereby increases the sensitivity of the described methodologies.

As most of the target drugs are lipophilic or hydrophobic, according to some of any of the embodiments described herein, the functional moiety is a lipophilic moiety.

Herein, the phrase “lipophilic moiety” describes a chemical moiety that can interact via hydrophobic interactions with a compound (e.g., a drug as described herein), depending on its chemical nature. It encompasses functional moieties that repel or fail to interact with water and/or other polar substances, tending to associate with non-polar substances or solvents. Without being bound by any particular theory, it is assumed that a lipophilic functional moiety forms a chemical (e.g., hydrophobic) interaction with a lipophilic drug. Non-limiting examples of hydrophobic chemical interactions that occur in hydrophobic environments include π-π interactions and van der Waals interactions.

According to some of any of the embodiments described herein, the functional moiety is selected capable of interacting with the drug by forming hydrophobic interactions therewith.

According to some of any of the embodiments described herein, the functional moiety is selected capable of interacting with a target drug by forming hydrophobic interactions therewith and as being capable of transferring electrons through the electrode. This may be effected, e.g., by means of a functional moiety that features polarity and/or aromaticity.

According to some of any of the embodiments described herein, the functional moiety comprises a hydrocarbon, as defined and described herein in any of the respective embodiments. In some of these embodiments, the hydrocarbon has at least 4, or at least 6, or at least 8, or at least 10, or at least 12, carbon atoms. In some embodiments, the hydrocarbon has from 4 to 30, or from 6 to 30, or from 8 to 30, or from 10 to 30, or from 12 to 30, carbon atoms, including any intermediate values and subranges therebetween.

In some of any of these embodiments, the hydrocarbon is substituted.

A hydrocarbon as described herein is or comprises one or more of an alkyl, alkenyl, alkynyl, cycloalkyl, aryl, and alkaryl, each being optionally substituted as described herein.

According to some of any of the embodiments described herein, the hydrocarbon is or comprises an aryl, as defined herein. In some of any of the embodiments described herein, the hydrocarbon is or comprises a substituted aryl. In some of any of the embodiments described herein, the hydrocarbon is or comprises a phenyl. In some of any of the embodiments described herein, the hydrocarbon is or comprises a substituted phenyl.

In some of any of the embodiments described herein, the hydrocarbon as described herein is or comprises a hydrocarbon chain, which can be saturated or unsaturated and can be linear or branched. In exemplary embodiments, the hydrocarbon is or comprises a linear hydrocarbon chain, as described herein. In exemplary embodiments, the hydrocarbon is or comprises a saturated linear hydrocarbon chain, as described herein. In some of any of the embodiments described herein, the hydrocarbon chain is of at least 6, or at least 8, or at least 10 carbon atoms in length, for example, of from 6 to 30, or from 6 to 20, or from 6 to 16 or from 8 to 30, or from 8 to 20, or from 8 to 16, carbon atoms in length. In some of any of the embodiments described herein, the hydrocarbon is an all-carbon hydrocarbon chain, and is devoid of interrupting heteroatoms, or is devoid of hydrophilic heteroatoms such as oxygen, nitrogen and sulfur.

According to some of any of the embodiments described herein, the hydrocarbon (e.g., a hydrocarbon chain and/or an aryl) features one or more substituents that impart polarity to the functional moiety and/or enhanced interactions with the drug.

In some of any of the embodiments described herein, the functional moiety comprises one or more polar group(s).

In some of any of the embodiments described herein, the functional moiety comprises one or more polar group(s), at least one of the one of more polar group(s) is independently an electron-withdrawing group as described herein.

In some of any of the embodiments described herein, the polar group(s) is a non-hydrophilic polar group(s). In some of any of the embodiments described herein, the functional moiety comprises a non-hydrophilic polar group(s) as described herein. In some of any of the embodiments described herein, the functional moiety comprises one or more non-hydrophilic electron-withdrawing group(s) as described herein. In some of any of the embodiments described herein, the functional moiety comprises one or more polar group(s), at least one, at least two, or all, of the one of more polar group(s) is independently a non-hydrophilic electron-withdrawing group(s) as described herein.

According to some of any of the embodiments described herein, the functional moiety comprises an electron-withdrawing group(s). According to some of any of the embodiments described herein, the functional moiety comprises one or more electron-withdrawing group(s).

As used herein and in the art, the phrase “electron-withdrawing group” (also referred to herein interchangeably as “electron-withdrawing moiety”) describes a chemical group or moiety that reduces electron density and pulls it away from adjacent atoms through inductive or resonance effects. Non-limiting examples include nitro (—NO₂), cyano (—CN), carbonyl groups (e.g., aldehydes, ketones, esters), trifluoromethyl (—CF₃), and halo (—F, —Cl—, —Br, —I).

In some of any of the embodiments described herein, the functional moiety is or comprises a hydrocarbon as described herein, substituted by one or more polar groups as described herein, preferably non-hydrophilic polar groups.

In some of any of the embodiments described herein, the functional moiety is or comprises a hydrocarbon as described herein, substituted by one or more fluoro-containing groups such as, for example, fluoro (—F), a trifluoroalkyl (e.g., trifluoromethyl, —CF₃), and/or a perfluorinated alkyl (e.g., pentafluoroethyl).

Without being bound by any particular theory, it is assumed that a fluoro substituent retains the hydrophobic (or non-hydrophilic) nature of the functional moiety while still providing polarity to the functional moiety, which facilitates interaction with the drug and/or electron transfer through the electrode.

In some of any of the embodiments described herein, the hydrocarbon as described herein is substituted by the one or more electron-withdrawing or polar group(s) as described herein. According to some of any of the embodiments described herein, the functional moiety comprises a hydrocarbon of at least 4 carbon atoms substituted by one or more electron-withdrawing or polar group(s) as described herein.

In some of any of the embodiments described herein, the hydrocarbon is or comprises an aryl substituted by one or more electron-withdrawing or polar group(s) as described herein. In some of any of the embodiments described herein, the hydrocarbon is or comprises a fluoro-substituted aryl. In some embodiments, the hydrocarbon is or comprises a fluoro-substituted phenyl. In some embodiments, the hydrocarbon is or comprises an aryl, such as phenyl, substituted by two, three, four or more fluoro-containing groups, as described herein. In some embodiments, the hydrocarbon is or comprises an aryl, such as phenyl, substituted by two, three, four or more fluoro substituents. In some embodiments, the hydrocarbon is or comprises a pentafluorophenyl.

In some of any of the embodiments described herein, the hydrocarbon is or comprises an alkaryl substituted by one or more electron-withdrawing or polar group(s) as described herein. In some of any of the embodiments described herein, the hydrocarbon is or comprises a fluoro-substituted alkaryl. In some embodiments, the hydrocarbon is or comprises a fluoro-substituted benzyl. In some embodiments, the hydrocarbon is or comprises a fluoro-substituted phenylethyl. In some embodiments, the hydrocarbon is or comprises a fluoro-substituted phenylpropyl. In some embodiments, the hydrocarbon is or comprises an alkaryl, such as benzyl, phenylethyl, or phenylpropyl, substituted by two, three, four or more fluoro-containing groups, as described herein. In some embodiments, the hydrocarbon is or comprises an alkaryl, such as benzyl, phenylethyl, or phenylpropyl, substituted by two, three, four or more fluoro substituents. In some embodiments, the hydrocarbon is or comprises a pentafluorophenylalkyl, wherein the alkyl is a lower alkyl, of 1, 2, 3 or 4 carbon atoms in length.

In some of any of the embodiments described herein, the hydrocarbon is a linear, saturated or unsaturated hydrocarbon chain substituted by one or more electron-withdrawing or polar group(s) as described herein.

In some of any of the embodiments described herein, the functional moiety comprises a linear saturated or unsaturated hydrocarbon chain substituted by two or more fluoro-containing groups, for example, two or more fluoro substituents.

According to some of any of the embodiments described herein, the hydrocarbon is or comprises a halo (e.g., fluoro)-substituted aryl (e.g., a fluoro-substituted phenyl), and/or a fluoro-substituted alkylene (e.g., of at least 4 carbon atoms in length; e.g., a perfluorinated alkyl chain).

According to some of any of the embodiments described herein, the functional moiety is or comprises a fluoro-substituted aryl such as phenyl, a fluoro-substituted alkaryl such as phenylalkyl and/or a fluoro-substituted alkylene of at least 4, at least 6, or at least 8, carbon atoms in length, as described herein in any of the respective embodiments.

In some of any of the embodiments described herein, the hydrocarbon is a perfluorinated alkyl. In some of any of the embodiments described herein, the hydrocarbon is a perfluorinated alkyl of at least 4, or at least 8, or at least 10, or at least 12, or at least 14, carbon atoms. In some of any of the embodiments described herein, the hydrocarbon is a perfluorinated alkyl of 14 carbon atoms.

In some of any of the embodiments described herein, the hydrocarbon is or comprises a perfluorinated aryl, such as, for example a pentafluorophenyl.

In some of any of the embodiments described herein, the functional moiety as described herein in any of the respective embodiments is covalently attached to the carbon electrode.

In some of any of the embodiments described herein, the functional moiety is attached to the carbon electrode via a linking moiety. According to some of any of the embodiments described herein, the linking moiety is selected capable of attaching to the carbon electrode and/or the functional moiety, preferably is such that does not impair the electron transfer through the electrode and/or the interaction with the drug.

Non-limiting examples of linking moieties include alkylenes, aryls, alkaryls, carbonyls, ethers, amines, sulfonates, phosphonates, silyls, and siloxanes. In some of any of the embodiments described herein, the linking moiety retains a non-hydrophilic or hydrophobic nature of the functional moiety, and can comprise, for example, an alkylene, an aryl, an alkaryl, a silyl, a siloxane and any combination thereof.

According to some of any of the embodiments described herein, the linking moiety is derived from a bi-functional substance that features one reactive group that is capable of forming a covalent bond with a reactive group on the surface of the carbon electrode, and another reactive group that is capable of attaching to a reactive group of the functional moiety. In some embodiments, the linking moiety is a substituent of the functional moiety that features a reactive group that can be covalently coupled with a reactive group on the carbon electrode surface.

According to some of any of these embodiments, the linking moiety is or comprises a silyl or a siloxane moiety. In some of any of the embodiments described herein, the functional moiety is attached to the carbon electrode via a siloxane linking moiety, or via one or more —O—Si— bond(s). According to some of these embodiments, a siloxane that is substituted by a functional moiety as described herein in any of the respective embodiments is reacted with reactive groups (e.g., hydroxy groups) that are intrinsically present or are generated on the electrode's surface, to thereby from a —O—Si— bond. According to some of these embodiments, a silyl that is substituted by a functional moiety as described herein in any of the respective embodiments and by a reactive group (such as a second reactive group as described herein) is reacted with reactive groups (e.g., hydroxy groups) that are intrinsically present or are generated on the electrode's surface, to thereby from a —O—Si— bond. According to some of these embodiments, a tetraorthosilicate that is substituted by a functional moiety as described herein in any of the respective embodiments is reacted with reactive groups (e.g., hydroxy groups) that are intrinsically present or are generated on the electrode's surface, to thereby from three —O—Si—O bonds.

In some of any of the embodiments described herein, the linking moiety is derived from an orthosilicate which can be optionally substituted by a functional moiety as described herein, or which features a reactive group that is capable of covalently attach to the functional moiety.

In some of any of the embodiments described herein, the linking moiety is derived from a silyl or a siloxane or an orthosilicate, as these are defined and described herein, substituted by one or more functional moieties as described herein.

In some embodiments, the functional moiety is a silyl, and is represented by Formula I:

-L-SiR1R2R3 Formula I
wherein L is a group linking the silicon atom to the electrode, or is a bond, one of R1, R2 and R3 is a functional moiety as described herein in any of the respective embodiments, and the others can be either a bond, a functional moiety as described herein, or a hydrophobic substituent such as an alkyl (e.g., lower alkyl). In exemplary embodiments, at least one of R1 and R2 is independently methyl and R3 is the functional moiety as described herein. In some of any of the embodiments described herein, R1 and R2 are each independently methyl and R3 is the functional moiety as described herein.

In some embodiments, the functional moiety is a siloxane, and is represented by Formula II:

—O—Si—R1R2R3 Formula II
wherein the oxygen atom is linked to surface groups of the electrode, at least one of R1, R2 and R3 is a functional moiety as described herein, and the others can be either an oxygen (—O—) that is also attached to the electrode, a functional moiety as described herein, and/or a hydrophobic substituent such as an alkyl (e.g., lower alkyl). In exemplary embodiments, at least one of R1 and R2 is independently methyl, and R3 is the functional moiety as described herein. In some of any of the embodiments described herein, R1 and R2 are each independently methyl and R3 is the functional moiety as described herein.

In some embodiments, the functional moiety is an orthosilicate, and is represented by Formula III:

—O—Si(OR1)(OR2)(OR3) Formula III
wherein the oxygen atom is linked to surface groups of the electrode, at least one of R1, R2 and R3 is a functional moiety as described herein, and the others can be either absent, such that a respective an oxygen is also attached to surface groups of the electrode, a functional moiety as described herein, and/or a hydrophobic substituent such as an alkyl (e.g., lower alkyl). In exemplary embodiments, and R3 is the functional moiety as described herein, and R1 and R2 are each absent, such that the functional moiety is linked to the carbon electrode via three —O—Si— bonds.

In some embodiments, the functional moiety is covalently attached to the electrode's surface by means of forming covalent bonds between a reactive group in a compound from which the functional moiety is derived and a compatible reactive group on the surface of the electrode. The functional moiety is the moiety formed upon such a covalent attachment of the compound to the reactive group on the electrode's surface.

For example, a functional moiety which is a siloxane, represented by Formula II (—O—Si—R1R2R3), can be generated from a corresponding siloxane R4-O—Si—R1R2R3, in which R4 can be hydrogen or, for example, alkyl, upon reacting the siloxane with reactive groups on the electrode's surface.

For example, a functional moiety which is a silyl, represented by Formula I (-L-Si—R1R2R3), wherein L is a bond, can be generated from a corresponding silane Z—Si—R1R2R3, in which Z can be a reactive group, which is a leaving group that upon reacting the silane with reactive groups on the electrode's surface, enables the formation of covalent bond with the electrode's surface, for example, upon a nucleophilic reaction with hydroxy groups on the electrode's surface.

Reactive groups on the electrode's surface are either intrinsic or can be generated upon a suitable treatment.

In some embodiments, a carbon fiber electrode as described herein is surface-modified so as to generate surface reactive groups (also referred to herein interchangeably as first reactive groups). Such a surface modification can be performed by, for example, attaching to intrinsic functional groups on the surface a bifunctional linker molecule, which comprises in one terminus thereof a reactive group that is capable of forming a bond with these intrinsic functional groups and in another terminus thereof a reactive group that can form a bond with the functional moiety (that is, with a reactive group of a compound that generates the functional moiety). Such a surface modification can alternatively comprise other treatments which generate surface reactive groups (e.g., by oxidation or reduction of intrinsic functional groups on the electrode's surface). Exemplary methods for chemically modifying electrodes include, but are not limited to, coating with an electron-conductive polymer film, covalent attachment of functional moieties, sol-gel technology, physical adsorption, and oxygen plasma treatment.

In some embodiments, the compound generating the functional moiety comprises, prior to being attached to the carbon electrode, a reactive group that can readily react with a (first) reactive group on the electrode's surface so as to form a covalent bond with the surface. Such a reactive group is also referred to herein interchangeably as a second reactive group.

In some of any of the embodiments described herein, the carbon electrode is a surface-modified electrode as described herein, which features surface hydroxy groups (also referred to herein interchangeably as first reactive groups). These surface reactive groups can participate in the covalent attachment of the functional moiety thereto, via a compatible reactive group in the compound generating the functional moiety (also referred to herein interchangeably as a second reactive group).

In some of any of the embodiments described herein, the (first) reactive group is hydroxy.

Selecting (second) reactive groups that are compatible with (first) reactive groups on the electrode of choice is within the capabilities of any person skilled in the art, particularly in view of the guidance provided herein. In some embodiments, a carbon fiber electrode features, or is modified so as to feature, hydroxy (first) reactive groups on its surface and the compound generating the functional moiety comprises a (second) reactive group capable of forming covalent bond with the free hydroxy groups on the electrode's surface. Exemplary such (second) reactive groups include, but are not limited to, halides and alkoxides, which can act as leaving groups so as to form an ether bond, carboxylic acids or esters, which can form an ester bond via esterification or trans esterification, as well as halosilanes, siloxanes and orthosilicates, which can form —Si—O— bonds, as exemplified herein by Formulae I and II, or —O—Si—O bonds, as described herein for Formula III.

According to some embodiments of the invention, the functional moiety is covalently attached to the electrode's surface via any one of the bonds described herein.

According to some embodiments of the invention, the functional moiety is covalently attached to the electrode's surface via one or more —O—Si— bond(s), and/or one or more —Si—O—Si bond(s), as described herein.

Free hydroxy groups on a carbon fiber electrode's surface can be generated, for example, by oxygen plasma treatment.

A carbon electrode featuring a functional moiety or a plurality of functional moieties as described herein in any of the respective embodiments, is also referred to herein interchangeably as a functionalized carbon electrode, a functionalized CF electrode, a modified carbon electrode, a modified CF electrode, a modified carbon paper electrode, a chemically-modified electrode, a chemically-modified carbon electrode, a chemically-modified CF electrode, and diversions and variations thereof.

According to an aspect of some embodiments of the present invention there is provided a process of preparing a modified carbon electrode as described herein, the process comprising coupling to a carbon electrode featuring a plurality of first reactive groups on at least a portion of its surface, as described herein, a compound featuring the functional moiety as described herein and a second reactive group that forms a covalent bond with the first reactive groups.

The first and second reactive groups are selected so as to be compatible with one another in forming a covalent bond therebetween, as described herein, and the coupling is effected under conditions (e.g., chemical reagents, solvent/s, chemical conditions such as pH and/or physical conditions such as heat, radiation, etc.) for promoting the formation of a covalent bond between the first and second reactive groups.

The first and second reactive groups, according to any of the embodiments described herein, can be such that interact via a chemical reaction such as nucleophilic substitution reaction, addition-elimination reaction, Diels-Alder reaction, and any other reaction that results in formation of a covalent bond. Reactions for forming covalent bonds, reactive groups participating in such reactions and conditions for promoting such reactions are readily recognized by those skilled in the art.

In some embodiments, the coupling is effected by contacting the carbon electrode and a compound from which the functional moiety derives, as described herein in any of the respective embodiments, optionally in the presence of a solvent, optionally at elevated temperatures (e.g., 50-150° C.).

In some of any of the embodiments described herein, the process further comprises, prior to the coupling, generating the first reactive group, or a plurality of first reactive groups, on at least a portion of the surface of the carbon electrode.

In some of any of the embodiments described herein, the first reactive groups are generated by oxidizing the surface of a portion thereof, to thereby generate, as a non-limiting example, hydroxy group and/or aldehydes or ketones or carboxylate groups in the electrode's surface or a portion thereof.

In some of any of the embodiments described herein, generating the first reactive groups comprises subjecting the carbon electrode to oxygen plasma treatment.

In some of any of the embodiments described herein, generating the first reactive groups comprises contacting the carbon electrode with hydroxide-containing solution.

Exemplary processes of surface modification of a carbon electrode and of preparing a modified carbon electrode as described herein is as described in the Examples section that follows.

As described herein, a method of determining a presence, type and/or amount of a drug is a gaseous sample, as described herein in any of the respective embodiments and any combination thereof, is effected by contacting a carbon electrode as described herein in any of the respective embodiments with the gaseous sample, and applying a potential to the carbon electrode.

In some of any of the embodiments described herein, the carbon electrode as described herein is usable for determining a presence, type and/or level of an electroactive drug in a gaseous sample, upon integrating the carbon electrode in an electrochemical system or device.

According to some of any of the embodiments described herein, the method comprises contacting the gaseous sample with a carbon electrode; applying potential to the electrode; and measuring the electrochemical parameter of the carbon electrode, wherein the electrochemical parameter is indicative of the presence and/or amount and/or type of the electroactive drug in the gaseous sample.

According to some of any of the embodiments described herein, the method comprises contacting the gaseous sample with a carbon electrode; applying potential to the electrode; and measuring the electrochemical parameter of the carbon electrode, wherein the electrochemical parameter is indicative of the presence and/or amount and/or type of the electroactive drug in the gaseous sample; wherein the carbon electrode is a gas-permeable electrode, and the contacting is by contacting at least a portion of the electrode with the gaseous sample.

Herein throughout, a presence, amount, and/or type of an electroactive drug is determined by a presence and/or level and/or pattern of an electrochemical parameter, such that upon contacting the gaseous sample with the carbon electrode, as these are described herein in any of the respective embodiments, a detectable change in the electrochemical parameter is formed if a drug is present in the gaseous sample, and is thereby indicative of a presence, type and/or amount of a drug in the sample.

In some of any of the embodiments described herein, the carbon electrode is selected capable of producing a measurable electrochemical parameter upon contacting a gaseous sample (i.e., in the presence and absence of the electroactive drug).

According to some of any of the embodiments described herein, contacting the sample with the carbon electrode is for a time period of no more than (up to) 1 minute, or no more than 50 seconds, or no more than 40 seconds, or no more than 30 seconds, or no more than 20 seconds, or no more than 10 seconds, including any intermediate values or subranges therebetween.

According to some of any of the embodiments described herein, a total operation time of the method is no more than (up to) 10 minutes, or no more than 9 minutes, or no more than 8 minutes, or no more than 7 minutes, or no more than 6 minutes, or no more than 5 minutes, or no more than 4.5 minutes, or no more than 4 minutes, or no more than 3.5 minutes, or no more than 3 minute, or no more than 2.5 minutes, or no more than 2 minute, or no more than 1.5 minutes, or no more than 1 minute, or even less, e.g., no more than 30 seconds, including any intermediate values or subranges therebetween.

In some embodiments, a method as described herein utilizes an electrochemical system or device, as described herein in any of the respective embodiments.

Herein throughout, an electrochemical system and electrochemical device are used interchangeably to describe a set-up that comprises an electrochemical cell as described herein into which the carbon electrode is integrated and which generates a measurable electrical parameter as described herein.

The following describes some embodiments of an electrochemical cell of the invention.

In some embodiments of the present invention, there is provided an electrochemical cell which comprises the carbon electrode as described herein in any of the respective embodiments and any combination thereof. The carbon electrode functions, and is also referred to herein, as a working electrode.

According to some of any of the embodiments described herein, the carbon electrode as described herein in any of the respective embodiments forms a part of an electrochemical cell. In some of any of the embodiments described herein, the electrochemical cell is operable by electrically connecting the electrode to a power source.

According to some of any of the embodiments described herein, the carbon electrode forms a part of an electrochemical cell and the electrochemical cell is operable by electrically connecting the electrode to a power source and thereby applying a potential to the electrode.

In some embodiments, the sensing electrode is electrically connectable to a power source, and the cell is configured such that when it is operated, at least a portion thereof, i.e., a portion thereof that has functional moieties as described herein covalently attached thereto, contacts the sample or at least a portion thereof.

In some embodiments, the carbon electrode is electrically connectable to other parts of a sensing system or device via electrically conducting wires, for example, conducting metal foils such as, but not limited to, Ni foils.

In some embodiments of the present invention, the electrochemical cell further comprises a reference electrode. Any commercially available or customarily-designed reference electrode is contemplated. In some embodiments, the reference electrode is an aqueous reference electrode. Exemplary usable reference electrodes include, but are not limited to, silver/silver chloride electrode (e.g., Ag/AgCl/saturated KCl electrode such as marketed by Metrohm®), a standard calomel (e.g., saturated calomel; SCE) electrode, a standard hydrogen electrode (SHE), a normal hydrogen electrode (NHE), a reversible hydrogen electrode (RHE), a copper-copper(II) sulfate electrode (CSE); a pH-electrode; a palladium-hydrogen electrode, a dynamic hydrogen electrode (DHE), and a mercury-mercurous sulfate electrode (MSE).

The reference electrode is also electrically connectable to a power source, and the cell is configured such that when it is operated, a potential difference (voltage) is applied between the sensing electrode and the reference electrode.

According to some of any of the embodiments described herein, the electrochemical cell further comprises a reference electrode and optionally an auxiliary electrode. In some embodiments, the electrochemical cell further comprises an auxiliary electrode. In some embodiments, the electrochemical cell follows a three-electrode design and further comprises an auxiliary electrode. Preferably, but not obligatory, the auxiliary electrode is a platinum (Pt) electrode, and is also referred to herein as a counter electrode. Any other auxiliary electrode, commercially available or customarily designed, is contemplated. Non-limiting examples include gold electrodes, carbon electrodes, and carbon/gold electrodes.

In some embodiments, the auxiliary electrode is electrically connectable to the carbon electrode.

A schematic presentation of an exemplary assembly of a three-electrode electrochemical cell 10 or cell 100 according to some embodiments of the present invention is presented in FIGS. 1A-B, respectively.

Electrochemical cell 10 (FIG. 1A) comprises a carbon (sensing) electrode 12 as described herein, which acts as a working electrode. Electrode 12 as described herein optionally features surface reactive groups as described herein (not shown). When the cell is operated, at least a portion of electrode 12 is in contact with the sample (not shown). The sample can be contacted with electrode 12 before it is integrated with cell 10 or when electrode 12 is already integrated with cell 10, e.g., by contacting an electrolyte 18 in which the sample is dissolved, or by contacting electrode 12 with the sample. Sensing electrode 12 is one-half of electrochemical cell 10. A reference electrode 22 is the other half of cell 10. A power source 20 is electrically connectable to sensing electrode 12 and reference electrode 22 by means of electrical wires 24. Power source 20 is configured to apply voltage between sensing electrode 12 and reference electrode 22, for example, by applying potential to one of the electrodes. Optionally, but not obligatory, cell 10 further comprises an auxiliary electrode 26, and a current measuring device 28, and device 28 is electrically connectable to sensing electrode 12 and auxiliary electrode 26.

Electrochemical cell 100 (FIG. 1B) comprises a carbon (sensing) electrode 12 as described herein, which acts as a working electrode. Sensing electrode 12 features functional moieties 16 as described herein at least on a portion of a surface thereof, and, optionally also features surface reactive groups as described herein (not shown). When cell 100 is operated, the portion of electrode 12 that features functional moieties 16 is in contact with the sample (not shown). The sample can be contacted with electrode 12 before it is integrated with cell 100 or when electrode 12 is already integrated with cell 100, e.g., by contacting an electrolyte 18 in which the sample is dissolved, or by contacting electrode 12 with the sample. Sensing electrode 12 is one-half of electrochemical cell 100. A reference electrode 22 is the other half of cell 100. A power source 20 is electrically connectable to sensing electrode 12 and reference electrode 22 by means of electrical wires 24. Power source 20 is configured to apply voltage between sensing electrode 12 and reference electrode 22, for example, by applying potential to one of the electrodes. Optionally, but not obligatory, cell 100 further comprises an auxiliary electrode 26, and a current measuring device 28, and device 28 is electrically connectable to sensing electrode 12 and auxiliary electrode 26.

For an electrochemical cell (e.g., cell 10 or cell 100) to operate, at least the carbon electrode (electrode 12) should be in contact with an electrolyte shown in FIGS. 1A-B as an electrolyte 18. The electrochemical cell (e.g., cell 10 or cell 100) can comprise an electrolyte (e.g., electrolyte 18, as exemplified in FIGS. 1A-B), or can comprise means (e.g., an inlet port; not shown in FIGS. 1A-B), for introducing the electrolyte to the cell, so as to contact at least the sensing electrode (e.g., sensing electrode 12).

An electrochemical cell according to the present embodiments can follow any of the designs known in the art, and can include one or more sensing electrode(s), and one or more of a reference electrode(s) and/or an auxiliary electrode(s). Exemplary designs include, without limitation, rotating disk-ring electrodes, ultramicro-electrodes, or screen printed electrodes.

The configuration of the components of electrochemical cell 10 or cell 100 as presented respectively in FIGS. 1A-B are for illustrative purpose only and are not to be regarded as limiting in any way.

Electrochemical cell 10 or electrochemical cell 100 can be, for example, in a form of a covered glass (or other inert material like Teflon or quartz) beaker, containing the sample solution in which the three electrodes are dipped. In some embodiments, any one of electrochemical cell 10 or electrochemical cell 100 is optionally a micro cell or a thin layer cell.

Electrochemical cell 10 or electrochemical cell 100 may further comprise means for mixing/stirring a sample with electrolyte 18 (not shown in FIGS. 1A-B).

Electrochemical cell 10 or electrochemical cell 100 may further comprise means for monitoring and/or controlling the temperature inside the cell (not shown in FIGS. 1A-B, respectively).

According to some of any of the embodiments described herein, the electrochemical cell further comprises an electrolyte.

As used herein and in the art, an electrolyte is an electrically conducting material or medium. An electrolyte can be solid or fluid, and can be used per se or when dissolved in a polar solvent, such as water. When dissolved in a solvent, it is referred to herein as an electrolyte solution. In the context of electrochemical cells, an electrolyte is also referred to as a background solution.

Herein throughout, the term “electrolyte” also encompasses an “electrolyte solution”.

In an electrochemical cell as described herein (e.g., electrochemical cell 10 or electrochemical cell 100, FIGS. 1A-B, respectively), at least the sensing electrode (e.g., sensing electrode 12) contacts the electrolyte (e.g., electrolyte 18) when the cell is operated. In some embodiments, all electrodes contact an electrolyte (e.g., electrolyte 18) when the cell is operated. In some embodiments, all electrodes contact the same electrolyte, as exemplified in FIGS. 1A-B, and in some embodiments, one or more of the electrodes contact an electrolyte different from the electrolyte in contact with the sensing electrode, and a membrane is interposed between the different electrolytes.

In some of any of the embodiments described herein, the electrolyte solution (e.g., electrolyte 18) is or comprises an aqueous solution.

In some of any of the embodiments described herein, the electrolyte solution (e.g., electrolyte 18) comprises a mixture of an aqueous solution and an organic solvent, preferably a water-miscible organic solvent. A volume ratio between an aqueous solution and an organic solvent can range, for example, from 10:1 to 1:10, or from 10:1 to 1:1, or from 5:1 to 1:1, or from 3:1 to 1:1, or from 5:1 to 3:1, or from 1:1 to 1:10, or from 1:1 to 1:5, or from 1:1 to 1:3, including any intermediate value and subranges therebetween. The aqueous solution can be water, per se, for example, distilled or double-distilled water, or can be a buffered aqueous solution, featuring pH of, for example, from 4 to 8, or from 5 to 8, or from 6 to 8.

Suitable organic solvents are preferably further characterized as being chemically compatible with (e.g., chemically inert to) the electrochemical cell or system as described herein. In some embodiments, the organic solvent is characterized as being chemically compatible with plastic and/or any other polymeric materials typically used for constructing electrochemical cells or systems.

An exemplary solvent is acetonitrile, although other solvents, such as, for example, dimethyl formamide, dimethylsulfoxide, propylene carbonate, ethanol, methanol, and any mixture thereof, are contemplated. Another exemplary solvent is ethanol.

In some of any of the embodiments described herein, the electrolyte solution comprises a quaternary ammonium salt. In some of any of the embodiments described herein, the quaternary ammonium salt is dissolvable in the electrolyte solution (e.g., in the organic solvent and/or an aqueous solution).

An organic quaternary ammonium salt can be represented by the Formula:

R₁R₂R₃R₄N⁺X⁻
wherein: R₁, R₂, R₃ and R₄ is each independently an alkyl, cycloalkyl or aryl, or alternatively, two or more form together a heterocyclic (heteroalicyclic or heteroaryl) ring; and X is an anion such as halide (e.g., chloride, bromide, iodide), perchlorate, borate, and any other acceptable anion.

The selection of the anion can be made such that it is inert to the electrochemical window of water, that is, the anion is preferably such that features a standard electrode potential higher than hydroxide.

In some embodiments, the anion is perchlorate.

In some embodiments, R₁, R₂, R₃ and R₄ is each independently an alkyl, and in some embodiments, each is independently an alkyl of from 1 to 6 carbon atoms. In some embodiments, R₁, R₂, R₃ and R₄ is each independently an alkyl of 4 carbon atoms.

In some of any of the embodiments described herein, the organic quaternary ammonium salt is soluble in the electrolyte solution as described herein in any of the respective embodiments.

Exemplary organic quaternary ammonium salts that are usable in the context of the present embodiments include commonly used phase transfer catalysts.

In some of any of the embodiments described herein, a concentration of the quaternary ammonium salt is lower than 0.5 M, or lower than 0.2 M, or lower than 0.1 M. In some embodiments, a concentration of the quaternary ammonium salt ranges from 1 to 100 mM, including any intermediate values and subranges therebetween. In some embodiments, a concentration of the quaternary ammonium salt is 50 mM.

Non-limiting examples of a quaternary ammonium salt is tetrabutylammonium perchlorate and tetrabutylammonium iodide, although other quaternary ammonium compounds are contemplated.

In some of any of the embodiments described herein, the carbon electrode forms a part of an electrochemical cell as described herein in any of the respective embodiments, or a part of a sensing system or device as described herein in any of the respective embodiments, and contacting the carbon electrode with the (e.g., gaseous) sample is effected by introducing the sample to the electrochemical cell, system or device, as described herein in any of the respective embodiments.

In some such embodiments, contacting the carbon electrode with the (e.g., gaseous) sample is effected by introducing the sample to the electrochemical cell, system or device, as described herein in any of the respective embodiments.

The sample can be introduced to the cell, system or device by means of a sample inlet, or by means of a pump, as described herein.

According to some of any of the embodiments described herein, the method comprises, prior to applying the potential, contacting the electrode with an electrolyte (e.g., the electrolyte as described herein in any of the respective embodiments).

According to some of any of the embodiments described herein, the method comprises, subsequent to contacting the electrode with the sample, contacting the electrode with an electrolyte.

In some of any of the embodiments described herein, a system or device as described herein further comprises means for introducing a sample to the electrochemical cell. When the electrode is not contacted with the sample prior to being assembled, the sample should be introduced to the cell such that it contacts the sensing electrode.

In some embodiments, the electrochemical system or device comprises means for introducing a sample to the electrochemical cell such that it contacts the sensing electrode.

In some embodiments, the electrochemical system or device comprises means for introducing a sample to the electrochemical cell such that it is mixed with or dissolved in the electrolyte solution.

In some embodiments, a sample is introduced to the electrochemical cell by means of an inlet port, referred to herein also as a sample inlet. In some embodiments, the inlet port is configured for introducing a gaseous sample to the cell.

In some of any of the embodiments described herein, the system is devoid of a sample inlet. This is enabled by the carbon fiber electrode, which is gas permeable and hence gas samples can enter the electrochemical cell therethrough.

In some of any of the embodiments described herein, the means for introducing a sample to the electrochemical cell include a pump or a pumping device. An exemplary pump is an air pump, which is usable when a sample in a gaseous form as described herein. The pump or the pumping device can be in contact with the CF microelectrode of the present embodiments, such that the pumped sample permeates through the electrode and is thus introduced to the cell or system. Alternatively, the pump or pumping device are in contact with the electrolyte, such that the pumped sample contacts or is mixed with the electrolyte.

In some of any of the embodiments described herein, the electrochemical system or device comprises an electrochemical cell as described herein in any of the respective embodiments and any combination thereof, and a means for contacting the gaseous sample with the carbon electrode.

According to some of any of the embodiments described herein, the means (i.e., for contacting the gaseous sample with the carbon electrode) comprises an air sampler. In some such embodiments, the means comprises an air pump. In some embodiments, the means comprises a breathing tube. In some embodiments, the means comprises an air-sampling device.

In alternative embodiments, the carbon electrode forms a part of an electrochemical cell as described herein in any of the respective embodiments, or a part of a sensing system or device as described herein in any of the respective embodiments, subsequent to contacting the carbon electrode and the sample as these are described herein in any of the respective embodiments.

In some embodiments, contacting the sensing electrode with the sample is effected by contacting a gas permeable electrode as described herein with a gaseous sample, for example, by means of an air pump as described herein, to thereby absorb at least a portion of the sample to the electrode, or at least a portion of the drug (e.g., in its gaseous state) to the electrode (optionally by means of the functional moiety attached to the carbon electrode) and/or to thereby provide a carbon electrode having at least a portion of the sample and/or the drug associated therewith (e.g., absorbed thereto). In some of these embodiments, the electrode is integrated with the electrochemical cell, contacted with the electrolyte and the cell is operated by applying voltage by means of the power source, as described herein in any of the respective embodiments.

FIGS. 5A and 5B present exemplary, non-limiting means for contacting a gaseous sample with a carbon electrode as described herein in any of the respective embodiments, prior to integrating it in an electrochemical cell as described herein.

Device 200 (FIG. 5A) comprises electrode 12 as described herein in any of the respective embodiments. Electrode 12 optionally features surface reactive groups as described herein (not shown) and may further feature functional moiety 16 as described herein (not shown). A component 220 represents means for contacting an air sample, as described herein, with electrode 12. In FIG. 5A, component 220 is in fluid communication with electrode 12 and is configured to generate a fluid communication between an environmental air sample and electrode 12, but means of an air pump (illustrated in FIG. 5A as a syringe, as a non-limiting example). Device 200 is operated by pumping air by component 220, to thereby have the air sample contacted with electrode 12 and provide electrode 12 having the sample or a portion thereof absorbed thereto. The electrode is thereafter integrated in an electrochemical cell as described herein, which is operated as described herein.

In some embodiments, not shown in FIG. 5A, device 200 can form a part of the electrochemical cell (e.g., cell 10 or 100, depending on the nature of electrode 12), such that electrode 12 already forms a part of the electrochemical cell when a sample is contacted with electrode 12 by means of component 220.

Device 210 (FIG. 5B) comprises electrode 12 as described herein in any of the respective embodiments. Electrode 12 optionally features surface reactive groups as described herein (not shown) and may further feature functional moiety 16 as described herein (not shown). A component 220 represents means for contacting a breath sample of a subject with electrode 12. In FIG. 5B, component 220 is in fluid communication with electrode 12 and is configured to generate a fluid communication between a breath sample of a subject and electrode 12. Device 210 is operated by having the subject breathing onto or into component 220, to thereby have the breath sample contacted with electrode 12 and provide electrode 12 having the sample or a portion thereof absorbed thereto. The electrode is thereafter integrated in an electrochemical cell as described herein, which is operated as described herein.

In some embodiments, not shown in FIG. 5B, device 210 can form a part of the electrochemical cell (e.g., cell 10 or 100, depending on the nature of electrode 12), such that electrode 12 already forms a part of the electrochemical cell when a sample is contacted with electrode 12 by means of component 220. In some embodiments, such a device is a breathalyzer as described herein.

According to some of any of the embodiments described herein, the electrochemical system or device comprises an electrochemical cell as described herein and means for contacting a gaseous sample with the electrode, as described herein in any of the respective embodiments and any combination thereof.

According to some of any of the embodiments described herein, the electrochemical cell or a system or device comprising same further comprises a gas outlet.

In some of any of the embodiments described herein, an electrochemical system or device as described herein is operable by assembling at least a carbon electrode as described herein and an electrolyte, and electric means for electrically connecting the carbon electrode to a power source, and optionally a reference and/or auxiliary electrode; introducing a sample into the electrochemical cell, by means that allow the sample to contact the electrode, as described herein; applying a potential to the carbon electrode, by means of a power source as described herein; and measuring an electrochemical signal or parameter that is indicative of an electrochemical reaction in which the drug participates.

In some of any of the embodiments described herein, an electrochemical system or device as described herein is operable by assembling a carbon electrode having a sample or a portion of a sample absorbed thereto or associated therewith as described herein and an electrolyte, and electric means for electrically connecting the carbon electrode to a power source, and optionally a reference and/or auxiliary electrode; applying a potential to the carbon electrode, by means of a power source as described herein; and measuring an electrochemical signal or parameter that is indicative of an electrochemical reaction in which the drug participates.

In some of any of the embodiments described herein, the electrochemical signal or parameter is an electrical current generated at the sensing electrode is response to the potential, and measuring the signal is effected by means of an electrical current measuring device. The measured current is indicative of a presence, type and/or level (e.g., amount, concentration) of a drug in the sample.

In some of any of the embodiments described herein, the electrochemical cell further comprises a device that measures a current generated at the sensing electrode, as a result of redox reactions occurring at or next to (in the vicinity of) a surface of the carbon electrode. In some embodiments, this device (i.e., the device that measures the current generated at the sensing electrode; e.g., an amperometer, a picoameter) is electrically connectable to the auxiliary electrode and the sensing electrode.

In some of any of the embodiments described herein, the electrochemical cell comprises a reference electrode and applying a potential is effected by applying voltage between the sensing electrode and the reference electrode.

The power source is configured to apply potential to the sensing electrode according to any known voltammetry method, as described in further detail hereinafter, in embodiments related to a sensing method.

In some embodiments, the power source is configured to apply a varying potential to the sensing electrode, and in some embodiments, the power source is configured to apply a linearly varying potential (as in linear sweep voltammetry); a staircase varying potential; a squarewave varying potential; or a pulse varying potential (normal pulse or differential pulse), as described in further detail hereinbelow.

In some embodiments, the power source is configured to apply differential pulse potential.

In some embodiments, the system is configured to determine a current generated in response to the varying potential, and in some embodiments, the system is configured for determining a change in the current generated at the sensing electrode, in response to the varying potential.

In some of any of the embodiments described herein, the system is configured to determine an electric current or a change in an electric current, compared to an electric current or a change in the electric current generated at the sensing electrode, in response to the varying potential, when a sample is not introduced to the electrochemical cell. Such data is also referred to herein as “background current” and in some embodiments, the system is configured to subtract the background current from the determined current or change in current.

In some embodiments, the system is operable in a differential pulse voltammetry mode and is configured to determine a change in an electrical current that is relative to a change in the potential (a derivative of the applied potential) in response to a change in the potential, as is known in the art.

Generally, but not necessarily, the system is configured for providing a voltammogram that presents values that are in line with the voltammetry methodology used.

In some embodiments the potential is a varying potential.

In some embodiments, measuring an electrochemical parameter is by voltammetry experiments. Voltammetry measurements are also referred to in the art as potentiostatic electrochemical analyses.

As known in the art, voltammetry experiments are conducted for obtaining information (e.g., presence, identity and/or level) of an analyte by measuring a generated current or a change in the current in response to application of a varying potential.

In order to obtain a quantitative measurement of an analyte (e.g., a drug as described herein) by potentiostatic electrochemical analysis, the amount of electrons used for the reduction/oxidation of the analyte should be monitored. In thermodynamic equilibrium the ratio of the redox-reactive species at the surface of the electrode can be obtained by Nernst equation:

$$E = {E}^{0} + \frac{2.3 RT}{nF} log ( \frac{{C}_{O}}{{C}_{R}} )$$

Where C_{O} is the concentration of the oxidized form, and C_{R} is the concentration of the reduced form, E is electrode potential, E⁰ is standard electrode potential, R is the gas constant

$$( 8.314 \frac{J}{K mol} ) ,$$

T is the temperature (Kelvin scale), n is the number of electrons participate in the redox reaction and F is the Faraday constant (96,487 coulombs).

The entire measured current is composed of Faradic currents and non-Faradic charging background current. The Faradic current obtained by the electrochemical reaction behaves according to Faraday's low, which means that 1 mole of redox active substance will involve a charge change of n×96,487 coulombs.

The information retrieved by voltammetry experiments, in its simplest form, is obtained as a voltammogram of I=f(E).

A voltammogram is a current versus potential curve used to describe the analyte's electrochemical reaction performed at the electrode as a result of the applied potential, and its derived current. It may have a complicated multi-stepped shape according to the complexity of the chemical reaction.

In some embodiments, and depending on the type of voltammetry used, the potential is varied continuously or stepwise or in pulses.

In some embodiments, the potential or varying potential applied to the sensing electrode is such that allows reduction or at least partial reduction of one or more nitro groups in a drug, typically to a corresponding fluoro group or groups.

Exemplary potentials that can be applied to a sensing electrode as described herein range from 0 to about −2 Volts.

Voltammetry experiments can be categorized as linear sweep voltammetry and cyclic voltammetry. Cyclic voltammetry is the process of electrochemical analysis in which the applied voltage is of a multi or mono-triangular shape. The resulting plot of current versus linear triangular potential scan of the working electrode is called cyclic voltammogram, while the plot of current versus linear potential scan of the working electrode is called linear sweep voltammogram. Cyclic voltammetry is usually the preliminary process used to determine the reduction potential of an analyte, the media's influence and the thermodynamics, as well as kinetics, of the electrochemical reaction.

In response to the triangular shaped potential, the measured current of the electrochemical cell that contained initially only the oxidized species, gradually increases up to a sharp peak at E_{p[red]}, followed by current decrease when most species adjacent to the electrode surface are reduced. When reversing the potential's direction, a gradual increase of current at the opposite direction ends in a sharp peak at E_{p[ox]}, where the chemical reaction proceeds to the opposite direction towards the oxidized form. When most species adjacent to the electrode surface are oxidized, the current decreases until the point of potential reverses, and so on.

Since an electrochemical reaction is located at the interface between the working electrode and the electrolyte solution, the reduced and oxidized species causing the sharp peaks of the voltammogram are concentrated to a narrow diffusive layer adjacent to the electrode. As a result, the shape of the curve's peak depends on the rate of diffusion. The peak's incline correlative to the concentration of electroactive particles on the electrode's surface, while the sharp decline depends solely on time, and results from the absence of electroactive particles near the surface due to limited diffusion.

In order to increase the sensitivity of voltammetric measurements, the share of the Faradic currents in the obtained voltammogram can be increased on the expense of the nonfaradaic background current. Such alterations are enabled by applying a series of short duration potential steps (each last for several milliseconds) in a technique termed “pulse voltammetry”. At the end of each potential step, two different current decay rates are obtained: sharp exponential decay to a negligible level is characteristic to the charging current, while slower decay is typical to the Faradic current. By recording the current's signal at the later regime, more of the signal is attributed to the Faradic current, while the contribution of the charging current is negligible. The differential pulse voltammogram is obtained from the subtraction of the pre-pulse current from the current that is obtained after the pulse is switched off, plotted against the applied potential. The corresponding sensitivity is thereby increased. The differential pulse voltammetry techniques vary by the shape of the applied potential waveform, and the current sampling technique.

Alongside increased sensitivity, differential pulse voltammetry allow the detection of two different analytes with similar redox potentials, by analysis of the peak's width according to the number of electrons that participate in their redox reaction. Exemplary values used for differential voltammetry measurements are 25-50 mV for current pulse amplitudes and 5 mV/second for the scan rate, while steeper amplitudes and faster scan rates are also contemplated.

In some of any of the embodiments described herein, the potential is a differential pulse varying potential.

In some of any of the embodiments described herein, the range of a varying potential ranges from −2 to +1 Volts, including any intermediate subranges therebetween.

In some of any of the embodiments described herein, an electrochemical parameter measured in a method as described herein is a change in electrical current, a change in electrical current relative to a derivative of the applied potential, or an absolute electrical current (upon subtracting the current in a same cell without a sample) although any other voltammogram is contemplated.

In some embodiments, the method is further effected by measuring the electrochemical parameter upon applying the potential to the carbon electrode, and in some embodiments, the electrochemical parameter is an electrical current generated at the carbon electrode or a change in the electrical current at the carbon electrode. A presence and/or level and/or pattern of the electrochemical parameter is indicative of a presence and/or level and/or type of the drug.

According to some of any of the embodiments described herein, applying the potential is by a cyclic voltammetry (CV) mode, a differential pulse voltammetry (DPV) mode, or a square-wave voltammetry (SWV) mode, preferably by cyclic voltammetry.

In some of any of the embodiments described herein, the method further comprises, prior to contacting the sample with the sensing electrode (e.g., prior to introducing the sample to the electrochemical cell), applying the potential, and measuring the electrochemical parameter, to thereby measure a background signal. A background signal is measured with the electrode before it is contacted with the sample and after it is contacted with the sample. In some embodiments, upon measuring the electrochemical parameter resulting from contacting the sensing system and the sample, the background signal is subtracted from the measured electrochemical parameter.

In some of any of the embodiments described herein, the method further comprises, subsequent to measuring the electrochemical parameter, applying an opposite potential to the sensing electrode, to thereby regenerate the electrode.

In some of any of the embodiments described herein, the measured electrochemical parameter is processed by a signal processor, as described herein in any of the respective embodiments, to thereby determine a presence, a type and/or a level of one or more drugs in the sample.

Determination of a change in the electrical current, according to any of the respective embodiments, can be performed by means of a device which is configured to process the received signals (e.g., the mode of the applied varying potential and corresponding generated current data) so as to provide a value or a set of values as desired (e.g., a change in electrical current relative to a derivative of the applied potential, or any other voltammogram). Such a device is also referred to herein as a signal processor or a hardware processor.

According to some of any of the embodiments described herein, the electrochemical cell is connectable to a hardware processor configured for receiving the signal generated by the electrode and processing the signal.

According to some of any of the embodiments described herein, the hardware processor is configured for transmitting the processed signal to a server computer at a remote location.

According to some of any of the embodiments described herein, the hardware processor is configured for processing the signal to determine presence, amount and/or type of the drug or portion thereof on the electrode and to transmit information pertaining to the determination to a server computer at a remote location.

According to some of any of the embodiments described herein, the hardware processor further comprises an indication unit in the form of a visual or an audio display.

In some embodiments, the signal processor is a data processor such as a computer configured for receiving and analyzing the signals. The signal processor extracts, from each generated signal or set of signals, a parameter (e.g., a voltammogram) that is indicative of an electrochemical reaction of a drug, and hence of a presence, type and/or level of the drug.

Herein throughout, by “type” of a drug it is meant an identity (a chemical composition) of a drug, if such is unknown when performing a method as described herein.

In some of any of the embodiments described herein, the signal processor is configured to construct a fingerprint of a drug, for example, a voltammogram obtained upon contacting with the electrode with the drug and applying a certain mode of a varying potential (e.g., a differential pulse potential).

In some of any of the embodiments described herein, the signal processor is configured to construct a database of fingerprints of a plurality of drugs, for example, a database of voltammograms obtained upon contacting a carbon electrode with a drug as those are described herein and applying a certain mode of a varying potential (e.g., a differential pulse potential). The database can include several voltammograms for each drug, each for a different mode and/or range and/or rate of application of the varying potential, and/or each for a different electrolyte.

In some of any of the embodiments described herein, the signal processor is configured to search a database of fingerprints of a plurality of drugs, for example, a database of voltammograms as described herein, for a fingerprint that matches a received fingerprint, and to identify accordingly the drug.

In some of any of the embodiments of the invention, the signal processor is configured to determine a level of an identified drug in a sample, by accessing and/or processing relevant data. Such data can include, for example, a calibration curve, e.g., of voltammograms, or of specific values obtained in voltammetry measurements (e.g., a reduction peak), obtained for varying concentrations of the identified drug, and stored on a computer readable medium. For example, the signal processor may access the calibration curve, search for a value (e.g., a reduction peak) that matches the value obtained upon operating the system, and identify a concentration of the identified drug that matches this value.

Alternatively or in addition, in some of any of the embodiments described herein, the data include a look-up table stored on a computer readable medium, which can be searched for values that match the measured value and are indicative of an identity and a level of a drug. Further alternatively, or in addition, the data include a predetermined relationship between the measured value and a level of the identified drug. For example, if such a predetermined relationship comprises a linear relationship, the signal processor can determine the level of an identified drug by means of extrapolation, based on the pre-determined relationship.

In some of any of the embodiments described herein, the electrochemical system or device as described herein further comprises an additional sensing carbon electrode, which is configured to generate an electrical signal upon contacting another drug. In some of these embodiments, the additional sensing carbon electrode forms a part of an additional electrochemical cell. Such a system is configured such that a sample is introduced therein and contacts both sensing electrodes. The generated electrical signals are thus indicative of the presence/absence and amount (if present) of both drugs.

In some of any of the embodiments described herein, such an electrochemical system or device further comprises a signal processor as described herein which is configured to identify each of the drugs, optionally, to further determine a level of each identified drug in a sample.

In some of any of the embodiments described herein, the additional sensing electrode is a carbon electrode which is modified so as to detect the additional drug.

In some such embodiments, the additional sensing electrode is optionally substituted by at least one functional group which allows (e.g., chemical) interaction with the additional drug(s).

In some of any of the embodiments described herein, an electrochemical system or device as described herein comprises a plurality (e.g., two, three or more) of CF electrodes as described herein, wherein at least one portion of the CF electrodes is modified so as to feature a first functional moiety and at least another portion of the CF electrodes features a second functional moiety which is different from the first functional moiety and/or do not feature a functional moiety, whereby both electrodes interact with a drug as described herein in any of the respective embodiments.

In some of these embodiments, the electrochemical system or device comprises three, four, or more portions of CF electrodes, each featuring a different functional moieties; or similar functional moieties having different substitution; or different functional moieties having different substitution, which interact with a drug as described herein.

In some of any of these embodiments, the electrochemical system or device comprises a plurality of electrochemical cells or a plurality of electrochemical half-cells, each being individually connectable to a power source, and, optionally, each being individually connectable to a device for measuring the electrochemical parameter as described herein.

In some of any of these embodiments, each of the measuring devices can independently be connected to a signal processor, or, alternatively, all measuring devices are connected to a signal processor as described herein in any of the respective embodiments.

Such an electrochemical system or device can generate different and defined fingerprints, and allows using, for example, a dedicated database or look-up table, for the identification of a drug (e.g., as described herein) or drugs based on such fingerprints database, according to the guidelines provided herein. In some of any of the embodiments described herein, determining the presence, amount, and/or type of the drug in the sample is performed based on a look-up table that correlates between the electrochemical parameter as described herein, or the group of electrochemical parameters generated with each electrode, and the presence and/or amount and/or type of the drug in the sample.

According to some of any of the embodiments described herein, the signal processor is configured to generate a visual or vocal sign or alert if a presence of a drug is detected.

According to some of any of the embodiments described herein, the signal processor is configured to generate a visual or vocal sign or alert if a presence of a drug is detected and a concentration of the drug (in its gaseous state) in the sample is such that is above a legal limit. For example, when the concentration of the drug in a breath sample of a subject is higher than the legal limit, the subject suspected as consuming the drug is not allowed to engage in certain activities (e.g., driving).

According to some of any of the embodiments described herein, the system or device further comprises a filter for preventing contact of a liquid or solid substance in the sample with the electrode, so as to assure that only the gaseous portion of a sample contacts the electrode. According to some of these embodiments, the sample is a breath sample, and the filter is for preventing a contact between the electrode and bodily fluids such as saliva, or any other bodily substances that are not gaseous.

According to some of any of the embodiments described herein, the system or device comprises a plurality of electrodes, each is individually as described herein, and/or a plurality of electrochemical cells (each is individually as described herein) comprising an electrode as described herein. According to some of these embodiments, one portion of the plurality of electrodes comprises a first functional moiety as described herein covalently attached thereto, and another portion of the plurality of electrodes comprises a second functional moiety as described herein covalently attached thereto, the second functional moiety being different from the first functional moiety, and/or another portion of the plurality of electrodes do not comprise a functional moiety as described herein. The non-modified electrodes are usable for detecting electroactive drugs that are less lipophilic, such as, for example, amphetamines, and each of the modified electrodes can be such that efficiently interact and result in efficient detection of a different electroactive drug.

Thus, an array of electrodes or electrochemical cells comprising same can be included in the system or device as described herein, which is capable of detecting various electroactive drugs in a single measurement.

According to an aspect of some embodiments of the present invention, there is provided a composition-of-matter comprising a carbon electrode as described herein in any of the respective embodiments and any combination thereof, optionally a chemically-modified carbon electrode as described herein, having a gaseous sample absorbed to or associated with at least portion of the electrode. The gaseous sample and/or components therein, for example, a drug as described herein in any of the respective embodiments, is absorbed or associated, for example, with a fibrous structure of the electrode, for example, by being absorbed to or entangled with the fibers that form the carbon electrode. In some embodiments, the electrode is a gas-permeable, fibrous carbon electrode as described herein in any of the respective embodiments. In some embodiments, the composition-of-matter is provided by contacting a gaseous sample with the electrode, for example, by means as described herein (e.g., as exemplified in FIGS. 5A-B).

In some of any of the embodiments described herein, the sensing system or device operates as a breathalyzer.

Herein, a “breathalyzer” describes a device used to measure the presence and/or level (concentration) of specific substances in a subject's breath. It encompasses the detection of drugs and/or alcohol, providing a means to determine the presence and level of these substances for various applications, e.g., law enforcement and medical diagnostics.

A common breathalyzer works by using an electrochemical cell to determine the concentration of a drug in the blood of a subject. When a person exhales into the breathalyzer's mouthpiece, the device collects a sample of deep lung air. This air sample is directed into the electrochemical cell within the device. When the drug's vapor from the breath sample enters the cell, an electrical current is generated, as described herein (e.g., for cell 10 or cell 100). The amount of current produced is proportional to the amount of the drug in the breath sample. The breathalyzer further comprises a device for measuring the generating current, as described herein, and a signal processor as described to calculate the concentration of a drug in the blood of the subject. The calculated concentration is then displayed on the device's screen.

The displayed reading can be used, for example, by law enforcement officers, to determine if the subject's drug level exceeds the legal limit for activities such as driving, thereby aiding in the enforcement of laws related to impaired driving.

An exemplary breathalyzer integrates cell 10 cell 100 shown in FIGS. 1A and 1, respectively, with a device 210 shown in FIG. 5B.

In some embodiments, a breathalyzer as described herein is for use in determining a presence and/or level of alcohol in a subject's blood.

In some embodiments, a breathalyzer as described herein is for use in determining a presence and/or level of a cannabinoid, as described herein, in a subject's blood.

In some embodiments, a breathalyzer as described herein is for use in determining a presence and/or level of an amphetamine, as described herein, in a subject's blood.

In some embodiments, a breathalyzer as described herein is for use in determining a presence and/or level of any of the electroactive drugs as described herein, in a subject's blood.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Herein throughout, the phrase “linking moiety” or “linking group” describes a group that connects two or more moieties or groups in a compound or a composition-of-matter (for example, connects between one or moieties of a compound and one or more moieties or groups on a surface of an electrode, as described herein). A linking moiety is typically derived from a bi- or tri-functional compound, and can be regarded as a bi- or tri-radical moiety, which is connected to two or three other moieties, via two or three atoms thereof, respectively.

Exemplary linking moieties include a hydrocarbon moiety or chain, optionally interrupted by one or more heteroatoms, as defined herein, and/or any of the chemical groups listed below, when defined as linking groups.

When a chemical group is referred to herein as “end group” it is to be interpreted as a substituent, which is connected to another group via one atom thereof.

Herein throughout, the term “hydrocarbon” collectively describes a chemical group composed mainly of carbon and hydrogen atoms. A hydrocarbon can be comprised of alkyl, alkene, alkyne, aryl, and/or cycloalkyl, each can be substituted or unsubstituted, and can be interrupted by one or more heteroatoms. The number of carbon atoms can range from 2 to 20, and is preferably lower, e.g., from 1 to 10, or from 1 to 6, or from 1 to 4. A hydrocarbon can be a linking group or an end group.

As used herein, the term “amine” describes both a —NR′R″ group and a —NR′— group, wherein R′ and R″ are each independently hydrogen, alkyl, cycloalkyl, aryl, alkaryl, heteroaryl, heteroalicylic, as these terms are defined hereinbelow.

The amine group can therefore be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

Alternatively, R′ and R″ can each independently be hydroxyalkyl, trihaloalkyl, alkenyl, alkynyl, amine, halide, sulfonate, sulfoxide, phosphonate, hydroxy, alkoxy, aryloxy, thiohydroxy, thioalkoxy, thioaryloxy, cyano, nitro, azo, sulfonamide, carbonyl, C-carboxylate, O-carboxylate, N-thiocarbamate, O-thiocarbamate, urea, thiourea, N-carbamate, O-carbamate, C-amide, N-amide, silyl, guanyl, guanidine and hydrazine.

The term “amine” is used herein to describe a —NR′R″ group in cases where the amine is an end group, as defined hereinunder, and is used herein to describe a —NR′— group in cases where the amine is a linking group or is or part of a linking moiety.

The term “alkyl” describes a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 6, or 1 to 4 carbon atoms (C(1-4) alkyl). The alkyl group may be substituted or unsubstituted. Substituted alkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, silyl, guanyl, guanidine and hydrazine.

The alkyl group can be an end group, as this phrase is defined hereinabove, wherein it is attached to a single adjacent atom, or a linking group, as this phrase is defined hereinabove, which connects two or more moieties via at least two carbons in its chain. When the alkyl is a linking group, it is also referred to herein as “alkylene” or “alkylene chain”.

Alkene (or alkenyl) and Alkyne (or alkynyl), as used herein, are an alkyl, as defined herein, which contains one or more double bond or triple bond, respectively.

The term “cycloalkyl” describes an all-carbon monocyclic ring or fused rings (i.e., rings which share an adjacent pair of carbon atoms) group where one or more of the rings does not have a completely conjugated pi-electron system. Examples include, without limitation, cyclohexane, adamantine, norbornyl, isobornyl, and the like. The cycloalkyl group may be substituted or unsubstituted. Substituted cycloalkyl may have one or more substituents, whereby each substituent group can independently be, for example, hydroxyalkyl, trihaloalkyl, cycloalkyl, alkenyl, alkynyl, aryl, heteroaryl, alkaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, silyl, guanyl, guanidine and hydrazine. The cycloalkyl group can be an end group, as this phrase is defined hereinabove, wherein it is attached to a single adjacent atom, or a linking group, as this phrase is defined hereinabove, connecting two or more moieties at two or more positions thereof.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofuran, tetrahydropyrane, morpholine, oxalidine, and the like. The heteroalicyclic may be substituted or unsubstituted. Substituted heteroalicyclic may have one or more substituents, whereby each substituent group can independently be, for example, hydroxyalkyl, trihaloalkyl, cycloalkyl, alkenyl, alkynyl, aryl, heteroaryl, alkaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, silyl, guanyl, guanidine and hydrazine. The heteroalicyclic group can be an end group, as this phrase is defined hereinabove, where it is attached to a single adjacent atom, or a linking group, as this phrase is defined hereinabove, connecting two or more moieties at two or more positions thereof.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted. Substituted aryl may have one or more substituents, whereby each substituent group can independently be, for example, hydroxyalkyl, trihaloalkyl, cycloalkyl, alkenyl, alkynyl, aryl, heteroaryl, alkaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, silyl, guanyl, guanidine and hydrazine. The aryl group can be an end group, as this term is defined hereinabove, wherein it is attached to a single adjacent atom, or a linking group, as this term is defined hereinabove, connecting two or more moieties at two or more positions thereof.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted. Substituted heteroaryl may have one or more substituents, whereby each substituent group can independently be, for example, hydroxyalkyl, trihaloalkyl, cycloalkyl, alkenyl, alkynyl, aryl, heteroaryl, alkaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, silyl, guanyl, guanidine and hydrazine. The heteroaryl group can be an end group, as this phrase is defined hereinabove, where it is attached to a single adjacent atom, or a linking group, as this phrase is defined hereinabove, connecting two or more moieties at two or more positions thereof. Representative examples are pyridine, pyrrole, pyrrolidone, oxazole, indole, purine and the like.

The term “alkaryl” describes an alkyl, as defined herein, which is substituted by one or more aryl or heteroaryl groups, as defined herein. An example of alkaryl is benzyl.

The term “halide”, “halogen” and “halo” describe fluorine, chlorine, bromine or iodine.

The term “haloalkyl” describes an alkyl group as defined above, further substituted by one or more halide.

The term “sulfate” describes a —O—S(═O)₂—OR′ end group, as this term is defined hereinabove, or an —O—S(═O)₂—O— linking group, as these phrases are defined hereinabove, where R′ is as defined hereinabove.

The term “thiosulfate” describes a —O—S(═S)(═O)—OR′ end group or a —O—S(═S)(═O)—O-linking group, as these phrases are defined hereinabove, where R′ is as defined hereinabove.

The term “sulfite” describes an —O—S(═O)—O—R′ end group or a —O—S(═O)—O— group linking group, as these phrases are defined hereinabove, where R′ is as defined hereinabove.

The term “thiosulfite” describes a —O—S(═S)—O—R′ end group or an —O—S(═S)—O— group linking group, as these phrases are defined hereinabove, where R′ is as defined hereinabove.

The term “sulfinate” describes a —S(═O)—OR′ end group or an —S(═O)—O— group linking group, as these phrases are defined hereinabove, where R′ is as defined hereinabove.

The term “sulfoxide” or “sulfinyl” describes a —S(═O)R′ end group or an —S(═O)— linking group, as these phrases are defined hereinabove, where R′ is as defined hereinabove.

The term “sulfonate” describes a —S(═O)₂—R′ end group or an —S(═O)₂— linking group, as these phrases are defined hereinabove, where R′ is as defined herein.

The term “S-sulfonamide” describes a —S(═O)₂—NR′R″ end group or a —S(═O)₂—NR′-linking group, as these phrases are defined hereinabove, with R′ and R″ as defined herein.

The term “N-sulfonamide” describes an R'S(═O)₂—NR″— end group or a —S(═O)₂—NR′-linking group, as these phrases are defined hereinabove, where R′ and R″ are as defined herein.

The term “disulfide” refers to a —S—SR′ end group or a —S—S— linking group, as these phrases are defined hereinabove, where R′ is as defined herein.

The term “oxo” as used herein, describes a (═O) group, wherein an oxygen atom is linked by a double bond to the atom (e.g., carbon atom) at the indicated position.

The term “thiooxo” as used herein, describes a (═S) group, wherein a sulfur atom is linked by a double bond to the atom (e.g., carbon atom) at the indicated position.

The term “hydroxyl” describes a —OH group.

The term “alkoxy” describes both an —O-alkyl and an —O-cycloalkyl group, as defined herein.

The term “aryloxy” describes both an —O-aryl and an —O-heteroaryl group, as defined herein.

The term “thiohydroxy” describes a —SH group.

The term “thioalkoxy” describes both a —S-alkyl group, and a —S-cycloalkyl group, as defined herein.

The term “thioaryloxy” describes both a —S-aryl and a —S-heteroaryl group, as defined herein.

The “hydroxyalkyl” is also referred to herein as “alcohol”, and describes an alkyl, as defined herein, substituted by a hydroxy group.

The term “cyano” describes a —C≡N group.

The term “isocyanate” describes an —N═C═O group.

The term “isothiocyanate” describes an —N═C═S group.

The term “nitro” describes an —NO₂ group.

The term “acyl halide” describes a —(C═O)R″″ group wherein R″″ is halide, as defined hereinabove.

The term “azo” or “diazo” describes an —N═NR′ end group or an —N═N— linking group, as these phrases are defined hereinabove, with R′ as defined hereinabove.

The term “carboxylate” as used herein encompasses C-carboxylate and O-carboxylate.

The term “C-carboxylate” describes a —C(═O)—OR′ end group or a —C(═O)—O— linking group, as these phrases are defined hereinabove, where R′ is as defined herein.

The term “O-carboxylate” describes a —OC(═O)R′ end group or a —OC(═O)— linking group, as these phrases are defined hereinabove, where R′ is as defined herein.

A carboxylate can be linear or cyclic. When cyclic, R′ and the carbon atom are linked together to form a ring, in C-carboxylate, and this group is also referred to as lactone. Alternatively, R′ and O are linked together to form a ring in O-carboxylate. Cyclic carboxylates can function as a linking group, for example, when an atom in the formed ring is linked to another group.

The term “thiocarboxylate” as used herein encompasses C-thiocarboxylate and O-thiocarboxylate.

The term “C-thiocarboxylate” describes a —C(═S)—OR′ end group or a —C(═S)—O— linking group, as these phrases are defined hereinabove, where R′ is as defined herein.

The term “O-thiocarboxylate” describes a —OC(═S)R′ end group or a —OC(═S)— linking group, as these phrases are defined hereinabove, where R′ is as defined herein.

A thiocarboxylate can be linear or cyclic. When cyclic, R′ and the carbon atom are linked together to form a ring, in C-thiocarboxylate, and this group is also referred to as thiolactone. Alternatively, R′ and O are linked together to form a ring in O-thiocarboxylate. Cyclic thiocarboxylates can function as a linking group, for example, when an atom in the formed ring is linked to another group.

The term “carbamate” as used herein encompasses N-carbamate and O-carbamate.

The term “N-carbamate” describes an R″OC(═O)—NR′— end group or a —OC(═O)—NR′-linking group, as these phrases are defined hereinabove, with R′ and R″ as defined herein.

The term “O-carbamate” describes an —OC(═O)—NR′R″ end group or an —OC(═O)—NR′— linking group, as these phrases are defined hereinabove, with R′ and R″ as defined herein.

A carbamate can be linear or cyclic. When cyclic, R′ and the carbon atom are linked together to form a ring, in O-carbamate. Alternatively, R′ and O are linked together to form a ring in N-carbamate. Cyclic carbamates can function as a linking group, for example, when an atom in the formed ring is linked to another group.

The term “carbamate” as used herein encompasses N-carbamate and O-carbamate.

The term “thiocarbamate” as used herein encompasses N-thiocarbamate and O-thiocarbamate.

The term “O-thiocarbamate” describes a —OC(═S)—NR′R″ end group or a —OC(═S)—NR′— linking group, as these phrases are defined hereinabove, with R′ and R″ as defined herein.

The term “N-thiocarbamate” describes an R″OC(═S)NR′— end group or a —OC(═S)NR′-linking group, as these phrases are defined hereinabove, with R′ and R″ as defined herein.

Thiocarbamates can be linear or cyclic, as described herein for carbamates.

The term “dithiocarbamate” as used herein encompasses S-dithiocarbamate and N-dithiocarbamate.

The term “S-dithiocarbamate” describes a —SC(═S)—NR′R″ end group or a —SC(═S)NR′— linking group, as these phrases are defined hereinabove, with R′ and R″ as defined herein.

The term “N-dithiocarbamate” describes an R″SC(═S)NR′— end group or a —SC(═S)NR′-linking group, as these phrases are defined hereinabove, with R′ and R″ as defined herein.

The term “urea”, which is also referred to herein as “ureido”, describes a —NR′C(═O)—NR″R′″ end group or a —NR′C(═O)—NR″— linking group, as these phrases are defined hereinabove, where R′ and R″ are as defined herein and R′″ is as defined herein for R′ and R″.

The term “thiourea”, which is also referred to herein as “thioureido”, describes a —NR′—C(═S)—NR″R′″ end group or a —NR′—C(═S)—NR″— linking group, with R′, R″ and R′″ as defined herein.

The term “amide” as used herein encompasses C-amide and N-amide.

The term “C-amide” describes a —C(═O)—NR′R″ end group or a —C(═O)—NR′— linking group, as these phrases are defined hereinabove, where R′ and R″ are as defined herein.

The term “N-amide” describes a R′C(═O)—NR″— end group or a R′C(═O)—N— linking group, as these phrases are defined hereinabove, where R′ and R″ are as defined herein.

An amide can be linear or cyclic. When cyclic, R′ and the carbon atom are linked together to form a ring, in C-amide, and this group is also referred to as lactam. Cyclic amides can function as a linking group, for example, when an atom in the formed ring is linked to another group.

The term “guanyl” describes a R′R″NC(═N)— end group or a —R′NC(═N)— linking group, as these phrases are defined hereinabove, where R′ and R″ are as defined herein.

The term “guanidine” describes a —R′NC(═N)—NR″R′″ end group or a —R′NC(═N)—NR″— linking group, as these phrases are defined hereinabove, where R′, R″ and R′″ are as defined herein.

The term “hydrazine” describes a —NR′—NR″R′″ end group or a —NR′—NR″— linking group, as these phrases are defined hereinabove, with R′, R″, and R′″ as defined herein.

The term “hydrazone” describes a —C(═O)—NR′—NR″R′″ end group or a C(═O)—NR′—NR″— linking group, as these phrases are defined hereinabove, with R′, R″, and R′″ as defined herein.

As used herein, the term “hydrazide” describes a —C(═O)—NR′—NR″R′″ end group or a —C(═O)—NR′—NR″— linking group, as these phrases are defined hereinabove, where R′, R″ and R′″ are as defined herein.

As used herein, the term “thiohydrazide” describes a —C(═S)—NR′—NR″R′″ end group or a —C(═S)—NR′—NR″— linking group, as these phrases are defined hereinabove, where R′, R″ and R′″ are as defined herein.

As used herein, the term “alkylene glycol” describes a —O—[(CR′R″)_{z}—O]_{y}—R′″ end group or a —O—[(CR′R″)_{z}—O]_{y}— linking group, with R′, R″ and R′″ being as defined herein, and with z being an integer of from 1 to 10, preferably, 2-6, more preferably 2 or 3, and y being an integer of 1 or more. Preferably R′ and R″ are both hydrogen. When z is 2 and y is 1, this group is ethylene glycol. When z is 3 and y is 1, this group is propylene glycol.

When y is greater than 4, the alkylene glycol is referred to herein as poly(alkylene glycol). In some embodiments of the present invention, a poly(alkylene glycol) group or moiety can have from 10 to 200 repeating alkylene glycol units, such that z is 10 to 200, preferably 10-100, more preferably 10-50.

The term “phosphonate” describes a —P(═O)(OR′)(OR″) end group or a —P(═O)(OR′)(O)— linking group, as these phrases are defined hereinabove, with R′ and R″ as defined herein.

The term “carbonyl” or “carbonate” as used herein, describes a —C(═O)—R′ end group or a —C(═O)— linking group, as these phrases are defined hereinabove, with R′ as defined herein. This term encompasses ketones and aldehydes.

The term “thiocarbonyl” as used herein, describes a —C(═S)—R′ end group or a —C(═S)-linking group, as these phrases are defined hereinabove, with R′ as defined herein.

The term “oxime” describes a ═N—OH end group or a ═N—O— linking group, as these phrases are defined hereinabove.

The term “cyclic ring” encompasses a cycloalkyl, a heretroalicyclic, an aryl (an aromatic ring) and a heteroaryl (a heteroaromatic ring), unless otherwise indicated.

Other chemical groups are to be regarded according to the common definition thereof in the art and/or in line of the definitions provided herein.

An “aryloxy” group describes both an —O-aryl and an —O-heteroaryl group, as defined herein.

A “silyl” describes a —SiR′R″R′″ end group or a —SiR′R″— linking group, as these phrases are defined hereinabove, whereby each of R′, R″ and R′″ are as defined herein.

A “siloxy” or “siloxane” or “alkoxysilane” describes a —Si(OR′)R″R′″ end group or a —Si(OR′)R″— linking group, as these phrases are defined hereinabove, whereby each of R′, R″ and R′″ are as defined herein.

A “silaza” describes a —Si(NR′R″)R′ end group or a —Si(NR′R″)— linking group, as these phrases are defined hereinabove, whereby each of R′, R″ and R′″ is as defined herein.

A “silicate” or “triorthosilicate” (derived from tetraorthosilicate) describes a —O—Si(OR′)(OR″)(OR′″) end group or a —O—Si(OR′)(OR″)—O— linking group, as these phrases are defined hereinabove, with R′, R″ and R′″ as defined herein.

Herein, the term “perfluorinated alkyl” refers to any alkyl as defined herein, that is substituted by fluorine atoms on essentially all available positions (i.e. wherein at least 80%, or 85%, or 90%, or 99.5%, and preferably at least 99.8%, of hydrogen atoms have been substituted by fluorine atoms). The perfluorinated alkyl group has 1 to 24 carbon atoms, and preferably at least 4 carbon atoms.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Experimental Methods

### Materials and Chemicals

Micro-carbon-fiber paper (0.18 mm thick) type SPECTRACARB™ 2050A-1050 was obtained from Engineered Fibers Technology, USA.

Parafilm PM996 was obtained from Alex Red, Israel.

Isolated THC, THCA, CBN and CBNA were obtained from Sigma Aldrich® and Tikun Olam.

Methamphetamine was obtained from Sigma Aldrich®.

Pentafluorophenylpropyldimethylchlorosilane (referred to herein as 5-fluorobenzene siloxane), dodecyldimethylchlorosilane (referred to herein as dodecane siloxane), and a perfluorinated C₁₄-alkyl-dimethylchlorosilane (referred to herein as siloxane-(CF₂)₁₄) were obtained from Gelest Ltd.

Millipore Mill-Q water (deionized water, 18 mega-ohm) was used in all experiments.

### Sensing Registration:

EmStat3+ (PalmSens BV, Netherlands) was used for linear sweep voltammetry and cyclic voltammetry measurements. EmStat3+ system is a compact and low-cost potentiostat with research grade capabilities. It has eight current ranges from 1 nA to 100 mA full scale, with a minimum resolution of 1 pA. It offers the most applicable electroanalytical measurement techniques.

### Construction of the Electrochemical Cell:

A conventional three-electrode cell (volume 3 ml) was assembled using platinum (Pt) electrode (0.4 cm²) as the counter electrode, a silver-silver chloride Ag—AgCl (deposition) wire (0.4 cm²) was used as a reference electrode, and micro-carbon-fibers (0.35 cm², 0.18 mm in diameter) as the working electrode.

The working electrode was prepared from micro-carbon-fiber paper (0.18×20×5 mm), with nickel foil (0.2×25×5 mm) used as a current collector (for forming electrical contact). The connections and the insulation of the contacts between the micro-carbon-fiber paper and the nickel foils were performed by Parafilm. The electrode and current collector were pressed at about 2 kg/cm² for 30 seconds at room temperature. The electrode was carefully washed with ethanol, rinsed with distilled water and dried at room temperature.

Before performing the electrochemical measurements, the micro-carbon-fibers paper electrode was washed with about 10 ml of isopropanol, and then with about 10 ml of deionized water.

The electrochemical measurements were performed as described below for each experiment.

Scanning electron microscope (SEA): measurements were performed using Quanta 200 FEG environmental scanning electron microscope.

### Example 1

### Electrochemical Detection of Cannabinoids in Solution

Electrochemical measurements were performed using an electrochemical cell as described in the Material and Methods section hereinabove, and as shown in FIG. 2. 50 mM tetrabutylammonium perchlorate in 70:30 water:acetonitrile was used as electrolyte (electrochemical background).

Measurements were performed in a cyclic voltammetry (CVA) mode; E_{begin}=−0.5 V, Eᵥₑᵣₜₑₓ₁=1.4 V, Eᵥₑᵣₜₑₓ₂=−0.5 V, scan rate=0.1 V/second, step=4 mV, number of scans=1.

The cyclic voltammetry of CBD (Cannabidiol) was measured at a concentration range of 8-64 ppm. Current peaks at 1.065 volts as a function of CBD concentration (linear fitting correlation R=0.996) are presented in FIGS. 3A-B, respectively.

Cyclic voltammetry of THC (delta9-terahydrocannabinol) was measured at a concentration range of 6-48 ppm. Current peaks at 1.31 volts as a function of THC concentration (linear fitting correlation R=0.996) are presented in FIGS. 3C-D, respectively.

Cyclic voltammetry of THCA (tetrahydrocannabinolic acid) was measured at a concentration range of 8-64 ppm. Current peaks at 1.42 volts as a function of THCA concentration (linear fitting correlation R=0.995) are presented in FIGS. 3E-F, respectively.

Cyclic voltammetry of CBN (cannabinol) was measured at a concentration range of 3-9 ppm. Current peaks at 1.48 volts as a function of CBN concentration (linear fitting correlation R=0.975) are presented in FIGS. 3G-H, respectively.

Cyclic voltammetry of CBNA (Cannabidiol acid) was measured at a concentration range of 8-64 ppm. Current peaks at 1.42 volts as a function of CBNA concentration (linear fitting correlation R=0.988) are presented in FIGS. 3I-J.

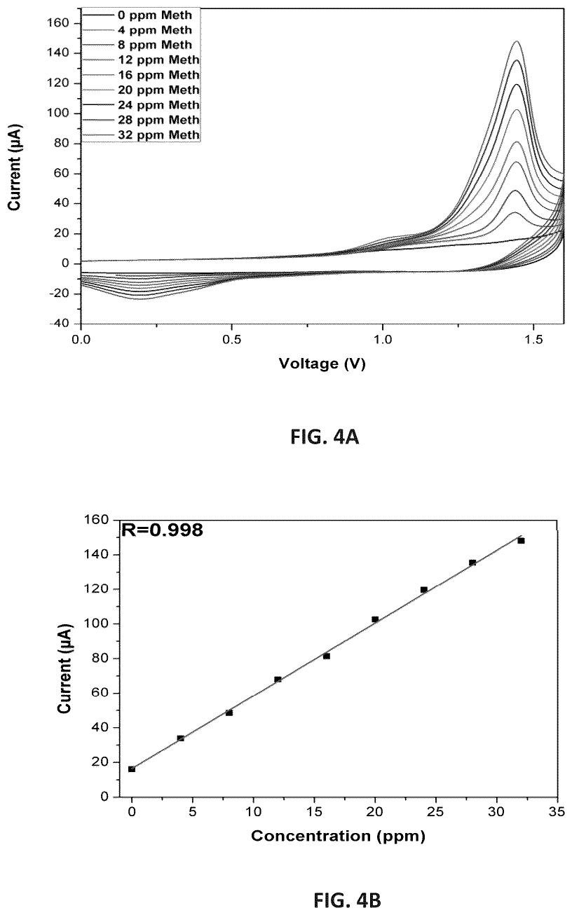

Cyclic voltammetry of Meth (Methamphetamine) was measured at a concentration range of 4-32 ppm. Current peaks at 1.44 volts as a function of Meth concentration (linear fitting correlation R=0.998) are presented in FIGS. 4A-B, respectively.

### Example 2

### Electrochemical Detection of Air Samples

In order to answer the need for a portable and robust electrochemical sensing system that can be applied in field conditions, a portable home-made air sampler was used. The air sampler is capable of pumping 10 liters of air per minute. This air sampling device is powered by rechargeable batteries and weighs 1 kg. The purpose of the air sampler system is to perform a collection of the drug sample from the air. The carbon electrode is fully air permeable, and enables air filtering and drug pre-concentration on the electrode surface.

A portion of an exemplary homemade air sampler is presented in FIG. 5A. The air sample is pumped through the silicon tubing and contacts the electrode, which is thereafter assembled into an electrochemical cell as described herein.

The collection of air from the drug sample surrounding (about 10 cm from the sample) was at a pace of 10 liters/minute for 5 seconds. The cyclic voltammetry mode was E_{begin}=−0.0 V, Eᵥₑᵣₜₑₓ₁=−0.4 V, Eᵥₑᵣₜₑₓ₂=1.6 V, scan rate=0.1 V/second, step=4 mV, number of scans=1.

The present inventors have recognized that in order to improve the sensing of cannabinoids in air samples, pre-concentration of the vapors onto the electrode's surface is desirable.

To this end, the carbon electrode was chemically modified by attaching thereto a moiety that on one hand does not affect electron transfer to the electrode during electrochemical detection, and on the other hand, is sufficiently lipophilic so as to absorb the lipophilic cannabinoids on its surface.

The following exemplary modifications were tested: 5-fluorobenzene siloxane, dodecane siloxane, and siloxane-(CF₂)₁₄, using the following general procedure.

In order to prepare the electrode's surface for the modification, it was first immersed in ethanol for 5 minutes and then washed with deionized water. Next, the micro-carbon-fiber electrode's surface was activated by exposure to 1% potassium hydroxide (weight percent in deionized water) for 10 minutes, followed by a gentle wash with deionized water. The micro-carbon-fiber electrode was then dried in a gentle stream of nitrogen and immediately oxidized by 30 W of O₂ plasma for 3 minutes at a pressure of 0.200 Torr. The pre-treated electrode was incubated with a respective siloxane derivative for 2 hours at 80° C. in a glass-sealed reactor. Finally, the modified electrode was washed with isopropanol and dried with a nitrogen stream.

The cyclic voltammetry data obtained for vapors of a solid CBD sample, collected during varying collection periods and the respective collection efficiency curves, using a bare carbon electrode and modified carbon electrodes, are presented in FIGS. 7A-H.

As can be seen, higher sensitivity was observed while using modified carbon electrodes, and a more pronounced improvement was seen with fluoro-containing modifications, particularly with fluoro-substituted phenyl.

FIG. 8 presents cyclic voltammetry data obtained for Meth vapors measured from a 2 mg sample (red) and for marijuana vapors after burning a 1.7 mg sample (in blue), compared to room air sampling recorded as a reference (black), using a 5-fluorobenzene-modified carbon paper microelectrode.

FIG. 9 presents cyclic voltammetry (CVA) data of human breath samples collected for a time period of 30 seconds before smoking and 10 minutes and 1 hour after smoking, cannabis, using an air collection device as depicted in FIG. 6 equipped with a fluorobenzene-modified carbon paper microelectrode (SPECTRACARB™ 2050A-1050, 0.35 cm²).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of detecting a presence, amount, and/or type of an electroactive drug in a gaseous sample, the method comprising:
contacting the sample with a carbon electrode;
applying potential to said electrode; and
measuring an electrochemical parameter of said carbon electrode, wherein said electrochemical parameter is indicative of a presence and/or amount and/or type of the electroactive drug in the sample; wherein
said carbon electrode is a gas-permeable electrode, and said contacting is by contacting at least a portion of the electrode with the gaseous sample,
wherein
said electroactive drug is lipophilic; and said carbon electrode features at least one functional moiety covalently attached thereto, said functional moiety being capable of interacting with said electroactive drug, and of allowing electron transfer to and/or through the electrode, said functional moiety being a lipophilic moiety.

2. The electrode of claim 1, wherein said carbon electrode is a carbon fiber microelectrode.

3. The method of claim 1, wherein said electroactive drug is a recreational drug.

4. The method of claim 1, wherein said electroactive drug is a cannabinoid or an amphetamine.

5. The method of claim 1, wherein said drug features a vapor pressure of no more than 0.1 Pa, or lower than 0.01 Pa, or lower, at room temperature; and/or wherein said gaseous sample comprises no more than 10 ng/liter of said drug in a gaseous state.

6. The method of claim 1, wherein said functional moiety comprises an electron-withdrawing group.

7. The method of claim 1, wherein said functional moiety comprises a non-hydrophilic electron-withdrawing group.

8. The method of claim 1, wherein said functional moiety comprises a hydrocarbon of at least 4 carbon atoms substituted by said at least one electron withdrawing group.

9. The method of claim 1, wherein said gaseous sample is an air sample.

10. The method of claim 9, wherein said air sample is of an environment of the drug.

11. The method of claim 10, wherein contacting the electrode with the gaseous sample is by means of a pump.

12. The method of claim 1, wherein said gaseous sample is a breath sample.

13. The method of claim 12, wherein contacting the gaseous sample with the electrode is by having the subject breathing in the vicinity of the electrode.

14. The method of claim 1, wherein said carbon electrode forms a part of an electrochemical cell and wherein the electrochemical cell is operable by electrically connecting said electrode to a power source.

15. The method of claim 14, wherein said electrochemical cell further comprises an electrolyte.

16. The method of claim 15, further comprising:
prior to applying said potential, contacting the electrode with said electrolyte; and/or
subsequent to contacting the electrode with the sample, contacting the electrode with an electrolyte.

17. The method of claim 14, wherein said electrochemical cell is connectable to a hardware processor configured for receiving said signal generated by said electrode and processing said signal.

18. An electrochemical system comprising an electrochemical cell which comprises a gas-permeable carbon electrode, and a means for contacting a gaseous sample with the carbon electrode.

19. The electrochemical system of claim 18, wherein said means comprises an air sampler, an air pump, and/or a breathing tube.

20. The electrochemical system of claim 18, being operable by contacting the gaseous sample with the carbon electrode to thereby provide a carbon electrode having the gaseous sample or a portion thereof absorbed thereto and integrating the electrode having the gaseous sample or a portion thereof absorbed thereto in said electrochemical cell and/or by introducing the gaseous sample to said electrochemical cell.
